(19) United States
(12) Patent Application Publication
(10) Pub. No.: US 2004/0259179 A1
(43) Pub. Date: Dec. 23, 2004

(54) Sterol markers as diagnostic tools in the prevention of atherosclerotic diseases and as tools to aid in the selection of agents to be used for the prevention and treatment of atherosclerotic disease

(76) Inventors: Gerd Assmann, Munster (DE); John R. Erbey II, Flemington, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/856,911

(22) Filed: May 28, 2004

Related U.S. Application Data
(60) Provisional application No. 60/474,438, filed on May 30, 2003.
(60) Provisional application No. 60/551,178, filed on Mar. 8, 2004.
(60) Provisional application No. 60/559,170, filed on Apr. 2, 2004.

(51) Int. Cl. C12Q 1/60; A61K 31/56

(52) U.S. Cl. 435/11; 514/170

(57) ABSTRACT

The present invention relates to methods for characterizing an individual's risk profile of developing a future cardiovascular disorder by measuring the level of sterols obtained from a individual. The present invention also includes methods of evaluating the likelihood of whether an individual will benefit from treatment with an agent for reducing risk of a future cardiovascular event, such as atherosclerosis, myocardial infarction and stroke.

### CROSS REFERENCE TO RELATED APPLICATIONS

The present application claims priority to U.S. Provisional Application Ser. Nos. 60/474,438, 60/551,178, and 60/559,170 filed May 30, 2003, Mar. 8, 2004 and Apr. 2, 2004, respectively, all of which are incorporated herein by reference in their entireties.

### BACKGROUND OF THE INVENTION

The present invention relates to use of a diagnostic test to evaluate the risk of vascular diseases such as atherosclerosis, myocardial infarction and stroke.

Vascular disease is a term that broadly encompasses all disorders of blood vessels including small and large arteries and veins and blood flow. As used herein, “vascular” comprises cardiovascular, cerebrovascular, peripheral vascular and combinations thereof.

The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease. Atherosclerotic coronary heart disease (CHD) represents the major cause for death and vascular morbidity in the western world. Other vascular diseases include vascular inflammation, cardiovascular events and stroke.

Role of Other Sterols

Elevated blood levels of the most common sterol of animal origin, cholesterol, are well known to be associated with development of atherosclerosis and with its major complication, myocardial infarction. In addition, rare genetic disorders characterized by extreme elevations of phytosterols, such as beta-sitosterolaemia and cerebrotendinous xanthomatosis also result in premature atherosclerosis (Björkhem I, Boberg K M. Inborn errors in bile acid biosynthesis and storage of sterols other than cholesterol. In: Scriver C R, Beaudet A L, Sly W S, Valle D, editors. The Metabolic and Molecular Bases of Inherited Disease on CD-ROM. 7 ed. New York: McGraw-Hill; 1997.) Individuals with beta sitosterolemia bear a mutation in the adenosine triphosphate binding cassette transporters ABCG5 or ABCG8, resulting in a failure to limit intestinal absorption of phytosterols (Hubacek J A, Berge K E, Cohen J C, Hobbs H H. Mutations in ATP-cassette binding proteins G5 (ABCG5) and G8 (ABCG8) causing sitosterolemia. Hum. Mutat. 2001;18:359-60; Berge K E, Tian H, Graf G A, Yu L, Grishin N V, Schultz J, Kwiterovich P, Shan B, Barnes R, Hobbs H H. Accumulation of dietary cholesterol in sitosterolemia caused by mutations in adjacent ABC transporters. Science 2000;290:1771-5) and leading to elevations in plasma sitosterol concentrations to between 250 and 1570 μmol/L. Since such patients generally have only modest elevations of low-density lipoprotein (LDL) cholesterol, the increased atherosclerosis is generally attributed to the elevation in phytosterols. While it would appear that extreme elevations of phytosterols predispose to atherosclerosis, the atherogenic role of the more modest phytosterol concentrations seen in the general population is less clear.

Plasma concentrations of phytosterols in the normal population vary between 1 and 25 μmol/L. A number of polymorphisms have been reported to contribute to the wide inter-individual distribution of plasma concentrations of plasma phytosterol level, indicating real differences in genetically determined sterol metabolism (Berge K E, von Bergmann K, Lutjohann D, Guerra R, Grundy S M, Hobbs H H, Cohen J C. Heritability of plasma noncholesterol sterols and relationship to DNA sequence polymorphism in ABCG5 and ABCG8. J. Lipid Res. 2002;43:486-94; Kempen H J, de Knijff P, Boomsma D I, van der Voort H A, Gevers Leuven J A, Havekes L. Plasma levels of lathosterol and phytosterols in relation to age, sex, anthropometric parameters, plasma lipids, and apolipoprotein E phenotype, in 160 Dutch families. Metabolism 1991;40:604-11.)

Humans are exposed to varying amounts of phytosterols, such as sitosterol, campesterol, stigmasterol and avenosterol, on a daily basis through the consumption of vegetable products (Salen G, Xu G, Tint G S, Batta A K, Shefer S. Hyperabsorption and retention of campestanol in a sitosterolemic homozygote: comparison with her mother and three control subjects. J. Lipid Res. 2000;41:1883-9). The most common dietary phytosterols, sitosterol and campesterol, are structurally related to cholesterol. While the presence of these two sterols in the diet is comparable to that of cholesterol, the absorption rate of sitosterol and campesterol in the intestine is markedly lower than the absorption rate of cholesterol (Heinemann T, Axtmann G, von Bergmann K. Comparison of intestinal absorption of cholesterol with different plant sterols in man. Eur. J. Clin. Invest 1993;23:827-31). Phytosterols taken up into the intestinal mucosa cells by ATP-cassette binding proteins (ABC) G5 and G8 are almost completely resecreted (Hubacek J A, Berge K E, Cohen J C, Hobbs H H. Mutations in ATP-cassette binding proteins G5 (ABCG5) and G8 (ABCG8) causing sitosterolemia. Hum. Mutat. 2001;18:359-60; Berge K E, Tian H, Graf G A, Yu L, Grishin N V, Schultz J, Kwiterovich P, Shan B, Barnes R, Hobbs H H. Accumulation of dietary cholesterol in sitosterolemia caused by mutations in adjacent ABC transporters. Science 2000;290:1771-5). In addition, ABC G5 and G8 are expressed in the liver and facilitate rapid excretion of phytosterols into the bile (Salen G, Ahrens E H, Jr., Grundy S M. Metabolism of beta-sitosterol in man. J. Clin. Invest 1970;49:952-67), so that their plasma concentrations are usually very low regardless of exposure (Vanhanen H T, Miettinen T A. Effects of unsaturated and saturated dietary plant sterols on their serum contents. Clin. Chim. Acta 1992;205:97-107).

Observational studies have indicated a potential clinical significance of modest elevations in phytosterol concentrations. Glueck et al. found elevated campesterol and sitosterol levels in hypercholesterolaemic patients with a family history of coronary heart disease and suggested that elevated plant sterols may be a risk factor for coronary heart disease independent of cholesterol (Glueck C J, Speirs J, Tracy T, Streicher P, Illig E, Vandegrift J. Relationships of serum plant sterols (phytosterols) and cholesterol in 595 hypercholesterolemic subjects, and familial aggregation of phytosterols, cholesterol, and premature coronary heart disease in hyperphytosterolemic probands and their first-degree relatives. Metabolism 1991;40:842-8). In a more recent study, 26 patients admitted for elective coronary artery bypass grafting with a history of coronary heart disease among their first-degree relatives had significantly higher levels of both sitosterol and campesterol compared to 27 coronary artery bypass patients without a family history of coronary heart disease (Sudhop T, Gottwald B M, von Bergmann K. Serum plant sterols as a potential risk factor for coronary heart disease. Metabolism 2002;51:1519-21). However, this result was not affected by adjustment for age, sex, triglycerides, total cholesterol, LDL cholesterol or high-density lipoprotein cholesterol. Finally, a group of middle-aged women with angiographically verified coronary artery disease was found to have higher levels of campesterol and sitosterol (and higher ratios of campesterol and sitosterol to cholesterol) than age-matched healthy women (Rajaratnam R A, Gylling H, Miettinen T A. Independent association of serum squalene and noncholesterol sterols with coronary artery disease in postmenopausal women. J. Am. Coll. Cardiol. 2000;35:1185-91). Although these studies suggest a correlation with hypercholesterolemia, it is uncertain whether associations observed in the studies are causal, due to short term changes, or interrelations with other factors, such as genetics and diet.

The assessment of an individual's risk of developing cardiovascular disease is a crucial, initial step in diagnosis and designing appropriate preventive or interventional treatment. Several tests have been developed to detect individuals at risk for developing cardiovascular disorders, such as screening tests for total and HDL cholesterol levels. However, these tests lack the sensitivity to differentiate degree of risk among individuals identified as having some moderate or elevated degree of risk. Also, these tests do not assess risk based on phytosterol levels. There is a need for a diagnostic test that can assist in identifying individuals at increased risk for cardiovascular disorders.

### SUMMARY OF THE INVENTION

One aspect of the present invention provides a method for characterizing a subject's risk profile of developing a future cardiovascular event, comprising:

(a) obtaining a level of at least one material selected from the group consisting of a phytosterol, a cholesterol precursor and a stanol in a subject having no history of clinically evident coronary heart disease prior to obtaining the level;

(b) comparing the level of the material to a predetermined material value (which can be a single value, multiple values, a single range or multiple ranges); and

(c) characterizing the subject's risk profile of developing a future cardiovascular event based upon the level of the material in comparison to the predetermined material value.

Another aspect of the present invention provides a method for characterizing a subject's risk profile of developing a future myocardial infarction, comprising:

(a) obtaining a level of at least one material selected from the group consisting of a phytosterol, a cholesterol precursor and a stanol in a subject;

(b) comparing the level of the material to a predetermined material value; and

(c) characterizing the subject's risk profile of developing a future myocardial infarction based upon the level of the material in comparison to the predetermined material value.

Another aspect of the present invention provides a method for characterizing a subject's risk profile of developing a future cardiovascular disorder associated with atherosclerotic disease, comprising:

(a) obtaining a level of at least one material selected from the group consisting of a phytosterol, a cholesterol precursor and a stanol in a subject;

(b) comparing the level of the material to a predetermined material value; and

(c) characterizing the subject's risk profile of developing a future cardiovascular disorder associated with atherosclerotic disease based upon the level of the material in comparison to the predetermined material value.

Another aspect of the present invention provides a method for characterizing a subject's risk profile of developing a future cardiovascular disorder associated with atherosclerotic disease, comprising:

(a) obtaining a level of at least one material selected from the group consisting of a phytosterol, a cholesterol precursor and a stanol in a subject;

(b) comparing the level of the material to a predetermined material value to establish a first risk value;

(c) obtaining a level of cholesterol in the subject;

(d) comparing the level of the cholesterol to a second predetermined cholesterol value to establish a second risk value; and

(e) characterizing the subject's risk profile of developing a future cardiovascular disorder associated with atherosclerotic disease based upon a combination of the first risk value and the second risk value.

Another aspect of the present invention provides a method for evaluating the likelihood that a subject will benefit from treatment with a sterol absorption inhibitor for reducing risk of a vascular disorder, comprising:

(a) obtaining a level of at least one material selected from the group consisting of a phytosterol, a cholesterol precursor and a stanol in a subject; and

(b) comparing the level of the material to a predetermined material value, wherein the level of the material in comparison to the predetermined material value is indicative of whether the subject will benefit from treatment with the sterol absorption inhibitor.

Another aspect of the present invention provides a kit for obtaining a level of at least one material selected from the group consisting of a phytosterol, a cholesterol precursor and a stanol in a subject and comparing the level of the material to a predetermined material value. The level of material can be obtained by any method well known to the skilled artisan, for example by gas chromatography as described below. The level can determined by measuring the level of material in a body fluid or tissue, such as blood, plasma, serum, lymph, saliva, urine and the like.

### BRIEF DESCRIPTION OF THE DRAWINGS

FIG. 1 shows a flow-chart of the procedure for selection of cases and controls in the PROCAM study.

FIG. 2 shows a bar graph of the distribution of sitosterol levels among the 354 controls in the PROCAM study.

FIG. 3 shows bar graphs of hazard ratios for the development of a coronary event for various risk factors on univariate analysis between cases and controls in the PROCAM study.

FIG. 3A shows bar graphs of hazard ratios for the development of a coronary event for various risk factors on univariate analysis between cases and controls in the PROCAM study (men only).

FIG. 4 shows a bar graph of hazard ratios for development of a coronary event for high and non-high sitosterol levels stratified according to LDL cholesterol among the cases and controls in the PROCAM study.

FIG. 5 shows a bar graph of hazard ratios for development of a coronary event for high and non-high sitosterol levels stratified according to global risk of coronary heart disease among the cases and controls in the PROCAM study.

FIG. 5A shows a bar graph of hazard ratios for development of a coronary event for high and non-high sitosterol to cholesterol ratios stratified according to global risk of coronary heart disease among the cases and controls in the PROCAM study.

FIG. 6 shows a bar graph demonstrating the percent change from baseline to endpoint phytosterol concentration in plasma samples from patients treated with ezetimibe, simvastatin or ezetimibe/simvastatin.

FIG. 7A shows a bar graph demonstrating percent change from baseline sitosterol concentration in plasma samples from patients treated with ezetimibe/simvastatin vs. simvastatin.

FIG. 7B shows a bar graph demonstrating percent change from baseline campesterol concentration in plasma samples from patients treated with ezetimibe/simvastatin vs. simvastatin.

FIG. 8 shows a bar graph demonstrating the percent change in cholesterol precursors/synthesis markers in plasma samples from patients treated with ezetimibe, simvastatin or ezetimibe/simvastatin.

FIG. 9A shows a bar graph demonstrating percent change from baseline lathosterol concentration in plasma samples from patients treated with ezetimibe/simvastatin vs. simvastatin.

FIG. 9B shows a bar graph demonstrating percent change from baseline desmosterol concentration in plasma samples from patients treated with ezetimibe/simvastatin vs. simvastatin.

FIG. 10 shows a bar graph demonstrating the percent change from baseline to endpoint non-cholesterol sterol concentration in relation to changes in LDL-C in plasma samples from patients treated with ezetimibe, simvastatin or ezetimibe/simvastatin.

FIG. 11 shows a bar graph demonstrating the percent change from baseline to endpoint phytosterol concentration in plasma samples from patients treated with ezetimibe, atorvastatin or ezetimibe/atorvastatin.

FIG. 12A shows a bar graph demonstrating percent change from baseline sitosterol concentration in plasma samples from patients treated with ezetimibe/atorvastatin vs. atorvastatin.

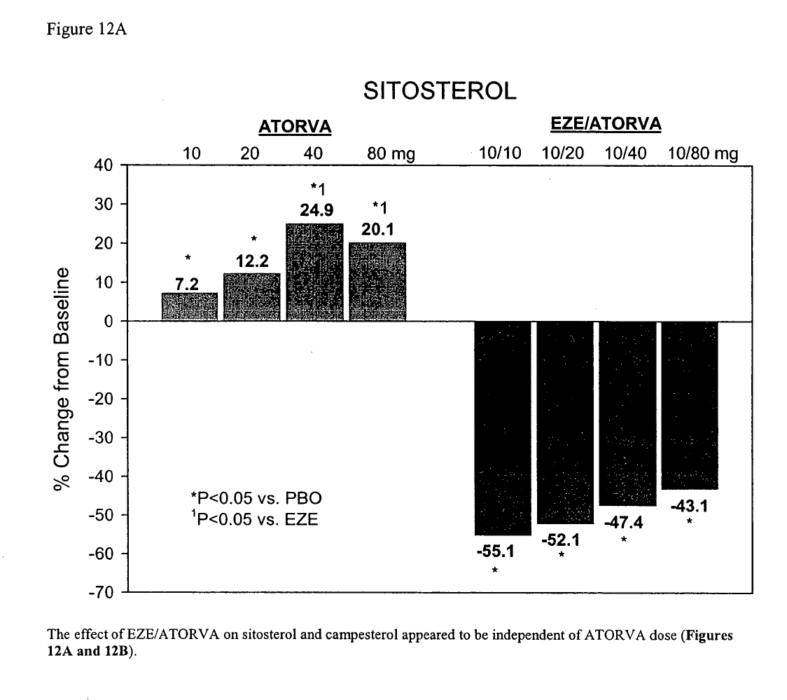

FIG. 12B shows a bar graph demonstrating percent change from baseline campesterol concentration in plasma samples from patients treated with ezetimibe/atorvastatin vs. atorvastatin.

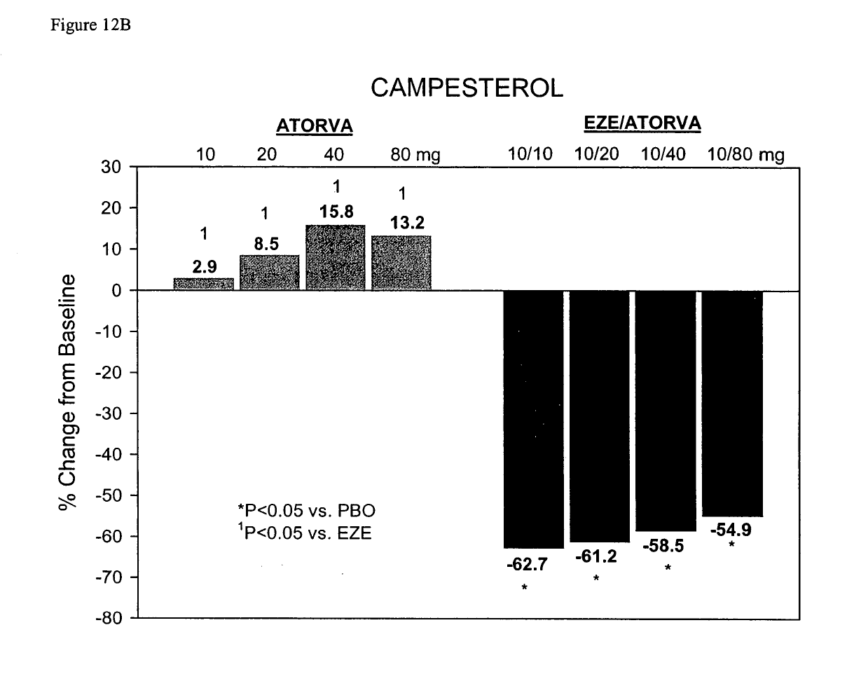

FIG. 13 shows a bar graph demonstrating the change in demonstrating the percent change from baseline to endpoint cholesterol precursors/synthesis markers in plasma samples from patients treated with ezetimibe, atorvastatin or ezetimibe/atorvastatin.

FIG. 14A shows a bar graph demonstrating percent change from baseline lathosterol concentration in plasma samples from patients treated with ezetimibe/atorvastatin vs. atorvastatin.

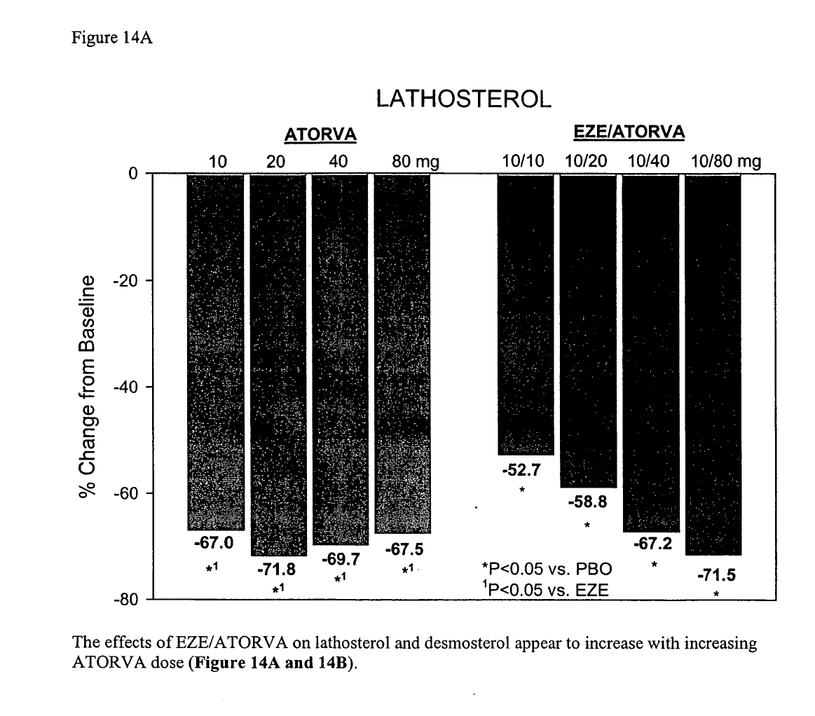

FIG. 14B shows a bar graph demonstrating percent change from baseline desmosterol concentration in plasma samples from patients treated with ezetimibe/atorvastatin vs. atorvastatin.

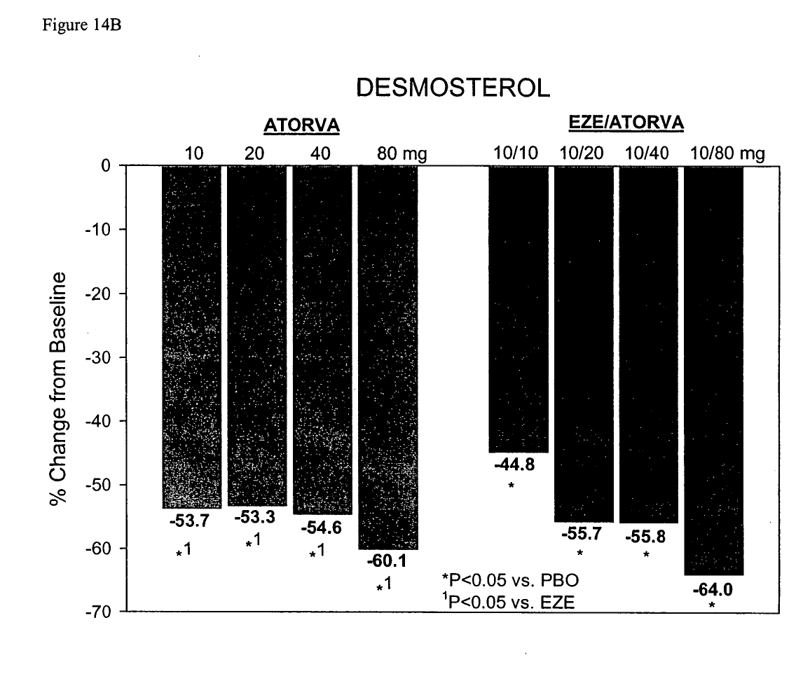

FIG. 15 shows a bar graph demonstrating the percent change from baseline to endpoint non-cholesterol sterols in relation to changes in LDL-C in plasma samples from patients treated with ezetimibe, atorvastatin or ezetimibe/atorvastatin.

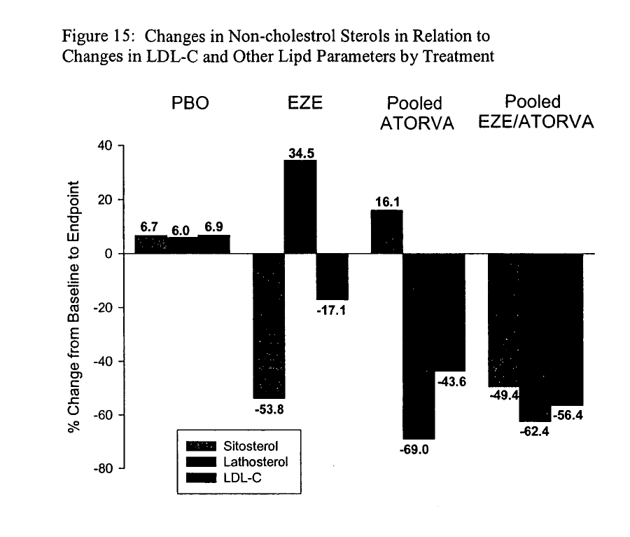

### DETAILED DESCRIPTION OF THE INVENTION

The present invention is based on the observation that levels of phytosterol, cholesterol precursor and/or stanol from a subject's tissue or fluid sample can be used to predict likelihood of future adverse cardiovascular events or disorders.

The methods of the present invention can be used to prevent or reduce the risk of an occurrence of a fatal or non-fatal cardiovascular event in patients having no history of clinically evident coronary heart disease prior to the initial administration of the compounds and treatments of the present invention, as well as patients having a history of clinically evident coronary heart disease. The phrase “cardiovascular event” includes, but is not limited to, fatal and non-fatal acute major coronary events, coronary revascularization procedures, peripheral vascular disease, stable angina and cerebrovascular insufficiency such as stroke. “Cardiovascular disorders associated with atherosclerotic disease” include myocardial infarction, stroke, angina pectoris and peripheral arteriovascular disease, but not venous thrombosis.

The phrase “acute major coronary event” includes fatal myocardial infarction, witnessed and unwitnessed cardiac death and sudden death occurring from 1 hour up to 24 hours after collapse, non-fatal myocardial infarction including definite acute Q-wave myocardial infarction, non-Q-wave myocardial infarction, and silent subclinical (remote) myocardial infarction, and unstable angina pectoris. As used herein, “myocardial infarction” includes both Q-wave and non-Q-wave myocardial infarction and silent subclinical (remote) myocardial infarction.

As used herein, “patient”, “subject” or “individual” means a mammal, such as a human, or other non-mammalian animal. An “apparently healthy” subject means a subject who has not previously had an acute adverse cardiovascular event, such as myocardial infarction. “Non-smoking” means a subject who is not a smoker at the time of evaluation. This includes subjects who have never smoked as well as subjects who have smoked in the past but presently no longer smoke.

The invention involves comparing the level in a sample of a subject's plasma, blood, serum, body fluid or tissue of at least one material selected from the group consisting of a phytosterol, a cholesterol precursor and a stanol with a predetermined value of that material.

In particular, the present invention provides a method for characterizing a subject's risk profile of developing a future cardiovascular event, comprising obtaining a level of at least one material selected from the group consisting of a phytosterol, a cholesterol precursor and a stanol in a subject having no history of clinically evident coronary heart disease prior to obtaining the level; comparing the level of the material to a predetermined material value (which can be a single value, multiple values, a single range or multiple ranges); and characterizing the subject's risk profile of developing a future cardiovascular event based upon the level of the material in comparison to the predetermined material value.

Practice of the present invention may involve obtaining the level of a potential marker of atherosclerosis, such as, e.g. phytosterol, in an individual subject. The level is compared to a predetermined value for the potential marker, where the comparison provides information as to whether the individual will gain any benefit from the administration of treatment with a sterol absorption inhibitor.

Non-limiting examples of suitable materials include compounds or complexes of phytosterols (such as sitosterol, campesterol, stigmasterol and avenosterol) and/or cholesterol precursors (such as lathosterol and desmosterol)) and/or stanols (including but not limited to 5α-stanols (such as cholestanol, 5 α-campestanol, 5α-sitostanol)).

The predetermined value can be a single value, such as a median or mean. It can be established based upon comparative groups, such as by defining a group in which the risk is double that of another group. It can be a range, for example the tested population can be divided into risk groups (low, medium, high) or risk quadrants.

The predetermined value can depend upon the population selected. For example, healthy non-smokers may have a value range different from that of smokers. In an embodiment of the invention, the predetermined value is obtained from healthy individuals.

In one embodiment, the predetermined material value is greater than about 4.5 micromoles per liter of plasma, blood, serum or tissue. In another embodiment, the predetermined material value is greater than about 5.0 micromoles per liter of plasma, blood, serum or tissue. In another embodiment, the predetermined material value is greater than about 5.25 micromoles per liter of plasma, blood, serum or tissue. In another embodiment, the predetermined material value is greater than about 7.0 micromoles per liter of plasma, blood, serum or tissue.

In one embodiment, the predetermined material (e.g. phytosterol, cholesterol precursor or stanol) value is greater than about 4.5 micromoles per liter of plasma, blood, serum or tissue. In another embodiment, the predetermined material value is greater than about 5.0 micromoles per liter of plasma, blood, serum or tissue. In another embodiment, the predetermined material value is greater than about 5.25 micromoles per liter of plasma, blood, serum or tissue. In another embodiment, the predetermined phytosterol value is greater than about 7.0 micromoles per liter of plasma, blood, serum or tissue.

In an embodiment of the invention, various known risk factors may be individually employed to evaluate risk. Analysis of risk may also comprise individual review of various other factors including, inter alia, age, sex, menopausal status, weight, obesity, individuals having a history of myocardial infarction, angina, stroke, or intermittent claudication, individuals with cardiovascular family history, smoking and various psychosocial factors. (Assmann et al., “Coronary Heart Disease:Reducing the Risk,” Circulation. 1999; 100:1930-1938.)

For example, individuals may be evaluated for levels of plasma total cholesterol, triglycerides, HDL cholesterol, and LDL cholesterol, blood pressure, glucose, etc, as measure of whether they will likely develop a future cardiovascular event. Increased levels of triglycerides, LDL, and HDL cholesterol level may be an indication of elevated risk. Levels of lipoprotein(s) exceeding 30 mg/dL are to confer increased risk. (Assmann et al., “Coronary Heart Disease: Reducing the Risk,” Circulation. 1999;100:1930-1938.)

In another embodiment, a quantitative estimate of risk is applied, where multiple factors are taken into consideration. Multiple risk factors may be applied using the algorithm, as disclosed below. These risk factors include age, LDL cholesterol, smoking, HDL cholesterol, systolic blood pressure, family history of premature myocardial infarction, diabetes mellitus, and triglycerides. The multiple logistic function has the form: I=1/[1+exp(−y)], where y=−12.3199+(age in years×0.1001)+(systolic blood pressure in mm Hg×0.0118)+(LDL cholesterol in mg/dL×0.0152)+(HDL cholesterol in mg/dL×−0.045)+(logₑ[triglyceride level in mg/dL]×0.3346)+(smoking behavior [no=0, yes=1]×0.9266)+(diabetes mellitus [no=0, yes=1]×0.4015)+(positive family history of myocardial infarction [no=0, yes=1]×0.4193)+(angina pectoris [no=0, yes=1]×1.319). The algorithm is available as an interactive program on the International Task Force for Coronary Heart Disease website (http://www.chd-taskforce.com). Application of this algorithm is shown in Example 1 in the Prospective Cardiovascular Munster (PROCAM) study.

A Cox proportional hazards model using various risk factors is generated, followed by a point scoring system based on the β-coefficients of the model. (Assmann G, Cullen P, Schulte H. Simple scoring scheme for calculating the risk of acute coronary events based on the 10-year follow-up of the prospective cardiovascular Munster (PROCAM) study. Circulation 2002;105:310-5). The accuracy of this point scoring scheme is comparable to coronary event prediction when the continuous variables themselves were used. The scoring system accurately predicted observed coronary events with an area under the receiver-operating characteristics curve of 82.4% compared with 82.9% for the Cox model with continuous variables.

The output of the PROCAM algorithm is expressed as the risk of a coronary event (definite fatal myocardial infarction, definite nonfatal myocardial infarction, or sudden coronary death) in percentage over 8 years. In an embodiment, the output of the algorithm may be divided into quintiles with the following cut-off points: first quintile, 0.91% in 8 years (0.11% per annum); second quintile, 0.92% to 1.40% in 8 years (0.12% to 0.18% per annum); third quintile, 1.41% to 3.65% in 8 years (0.18% to 0.46% per annum); fourth quintile, 3.66% to 7.60% in 8 years (0.46% to 0.95% per annum); and fifth quintile, >7.60% in 8 years (>0.95% per annum).

In another aspect of the present invention, kits are provided which are specific for or have appropriate sensitivity with respect to predetermined values selected on the basis of the invention. Such kits would have specified cut-offs and instructions or printed material for characterizing risk based upon outcome of the assay or measurement.

Another useful method for calculating risk is discussed in W. Castelli et al., “Incidence of Coronary Heart Disease and Lipoprotein Cholesterol Levels”, 256 JAMA 20 (Nov. 28, 1986) 2835-2838. Other useful methods for calculating risk are well known to those skilled in the art.

As discussed above, the invention provides methods for evaluating whether a subject might benefit from treatment with a sterol absorption inhibitor for reducing risk of a future vascular disorder. This method provides benefits in patient treatment and for clinical development of therapeutics. Physicians select treatments based upon the expected benefit to the patient. The present invention permits selection of individuals who are more likely to benefit from intervention, thereby aiding the physician to select a treatment in which benefit is more likely. Also, the present invention can assist in selecting subjects for clinical trials by defining a population having a higher likelihood of obtaining a net benefit from treatment.

The methods of the present invention is also useful for determining whether an apparently healthy individual may develop a future cardiovascular event.

In another embodiment, the invention provides a method for characterizing a subject's risk profile of developing a future cardiovascular disorder associated with atherosclerotic disease comprising obtaining a level of cholesterol in the subject and comparing the level of the cholesterol to a predetermined cholesterol value to establish a risk value, and characterizing the subject's risk profile of developing a future cardiovascular disorder associated with atherosclerotic disease based upon a combination of the risk value associated with phytosterol, cholesterol precursor and/or stanol levels, as described above and the risk value associated with cholesterol.

It has been discovered that phytosterol, cholesterol precursor and/or stanol levels can have predictive value independent of other known predictors of future adverse cardiovascular disorders. The present invention does not merely duplicate measurements that could have been made using other predictors, but can be indicative of risk for cardiovascular events and can be additive to previous predictors of increased risk of coronary events, such as high LDL cholesterol.

One aspect of the present invention provides new diagnostic tests for predicting risk of a vascular disorder or disease, including but not limited to atherosclerotic disorders such as myocardial infarction, stroke and peripheral arterial disease, as well as determining the likelihood that certain subjects will benefit to a greater or lesser extent from the use of sterol absorption inhibitors.

Non-limiting examples of suitable sterol absorption inhibitors and methods of making the same include those disclosed in U.S. Pat. Nos. 5,767,115; 5,846,966; 5,756,470, 5,698,548; 5,624,920; 5,656,624; 5,688,787; 5,688,990, 5,631,365, 6,207,822 and 6,627,757, each of which is incorporated herein by reference, for example:

(a) a sterol absorption inhibitor represented by Formula (I):

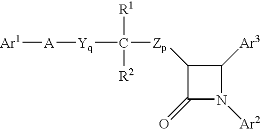

or isomers thereof, or pharmaceutically acceptable salts or solvates of the compounds of Formula (I) or of the isomers thereof, wherein in Formula (I):

Ar¹ is R³-substituted aryl;

Ar² is R⁴-substituted aryl;

Ar³ is R⁵-substituted aryl;

Y and Z are independently selected from the group consisting of —CH₂—, CH(lower alkyl)- and —C(di-lower alkyl)-;

A is —O—, —S—, —S(O)— or —S(O)₂—;

R¹ is selected from the group consisting of —OR⁶, —O(CO)R⁶, —O(CO)OR⁹ and —O(CO)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂—CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl;

(b) a sterol absorption inhibitor represented by Formula (II):

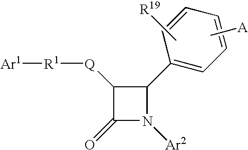

or isomers thereof, or pharmaceutically acceptable salts or solvates of the compounds of Formula (II) or of the isomers thereof, wherein in Formula (II):

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group;

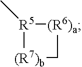

R¹ is selected from the group consisting of

—(CH₂)_{q}—, wherein q is 2-6, provided that when Q forms a spiro ring, q can also be zero or 1;

—(CH₂)e-G-(CH₂)ᵣ—, wherein G is —O—, —C(O)—, phenylene, —NR⁸— or —S(O)₀₋₂-e is 0-5 and r is 0-5, provided that the sum of e and r is 1-6;

—(C₂-C₆ alkenylene)-; and

—(CH₂)_{f}—V—(CH₂)_{g}, wherein V is C₃-C₆ cycloalkylene, f is 1-5 and g is 0-5, provided that the sum of f and g is 1-6;

R⁵ is

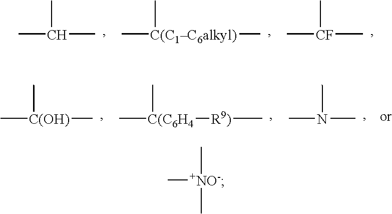

R⁶ and R⁷ are independently selected from the group consisting of —CH₂, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)-group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be:

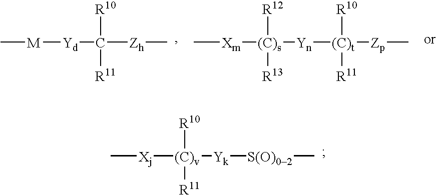

M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴ R⁵;

R¹¹ and R¹³ are independently selected from the group consisting of hydrogen, (C₁-C₆)alkyl and aryl; or R¹⁰ and R¹¹ together are ═O, or

R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or

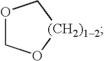

and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

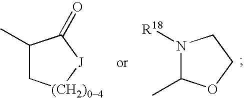

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)_{1-1O}CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl (C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen,

(C₁-C₆)alkyl, aryl and aryl-substituted (C₁-C₆)alkyl; R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy;

(c) a sterol absorption inhibitor represented by Formula (III):

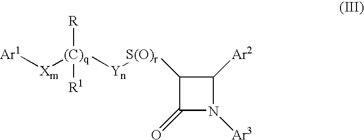

or isomers thereof, or pharmaceutically acceptable salts or solvates of the compounds of Formula (III) or of the isomers thereof, wherein in Formula (III):

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(di-lower alkyl)-;

R is —OR⁶, —O(CO)R⁶, —O(CO)OR⁹ or —O(CO)NR⁶R⁷;

R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of −OR⁶, —O(CO)R⁶, —O(CO)OR⁹, —O(CH₂)₁₋₅OR⁶, —O(CO)NR⁶R⁷, —NR⁷R⁸, —NR⁶(CO)R⁷, —NR⁶(CO)OR⁹, —NR⁶(CO)NR⁷R⁸, —NR⁶SO₂R⁹, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁶, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂ and halogen;

(d) a sterol absorption inhibitor represented by Formula (IV):

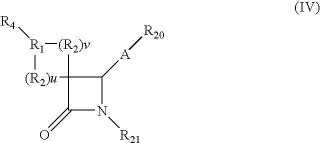

or isomers thereof, or pharmaceutically acceptable salts or solvates of the compounds of Formula (IV) or of the isomers thereof, wherein in Formula (IV):

R₁ is

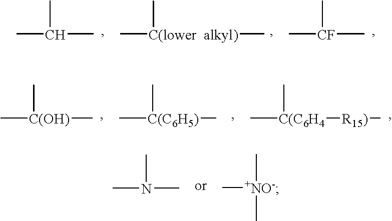

R₂ and R₃ are independently selected from the group consisting of:

—CH₂—, —CH(lower alkyl)-, —C(di-lower alkyl)-, —CH═CH— and —C(lower alkyl)=CH—; or

R₁ together with an adjacent R₂, or R₁ together with an adjacent R₃, form a —CH═CH— or a —CH═C(lower alkyl)- group;

u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;

R₄ is selected from B—(CH₂)ₘC(O)—, wherein m is 0, 1, 2, 3, 4 or 5;

B—(CH₂)_{q}—, wherein q is 0, 1, 2, 3, 4, 5 or 6;

B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;

B—(C₂-C₆ alkenylene)-;

B—(C₄-C₆ alkadienylene)-;

B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;

B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;

B—(CH₂)ₜ—V—(C₂-C₆ alkenylene)- or

B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or

R₁ and R₄ together form the group

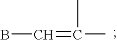

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

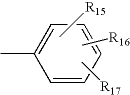

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and

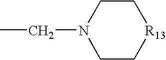

for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;

R₇ is 1-3 groups independently selected from the group consisting of lower alkyl, lower alkoxy, —COOH, NO₂, —N(R₈)(R₉), OH, and halogeno;

R₈ and R₉ are independently selected from H or lower alkyl;

R₁₀ is selected from lower alkyl, phenyl, R₇-phenyl, benzyl or R₇-benzyl;

R₁₁ is selected from OH, lower alkyl, phenyl, benzyl, R₇-phenyl or R₇-benzyl;

R₁₂ is selected from H, OH, alkoxy, phenoxy, benzyloxy,

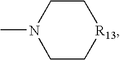

N(R₈)(R₉), lower alkyl, phenyl or R₇-phenyl;

R₁₃ is selected from —O—, —CH₂—, —NH—, —N(lower alkyl)- or —NC(O)R₁₉;

R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;

R₁₉ is H, lower alkyl, phenyl or phenyl lower alkyl; and

₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above;

(e) a sterol absorption inhibitor represented by Formula (VA) or Formula (VB):

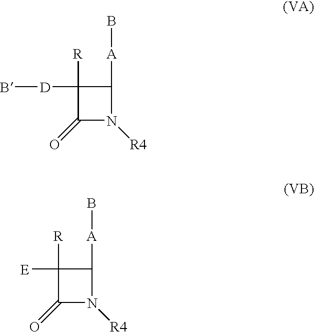

or isomers thereof, or pharmaceutically acceptable salts or solvates of the compounds of Formula (VA) or (VB) or of the isomers thereof, wherein in Formulae (VA) or (VB):

is —CH═CH—, —CC— or —(CH₂)ₚ— wherein p is 0, 1 or 2;

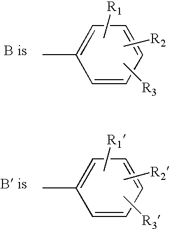

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3; R₁, R₂, R₃, R_{1′}, R_{2′}, and R_{3′} are independently selected from the group consisting of hydrogen, lower alkyl, lower alkoxy, carboxy, NO₂, NH₂, OH, halogeno, lower alkylamino, di-lower alkylamino, —NHC(O)OR₅, R₆O₂SNH— and —S(O)₂NH₂;

R₄ is

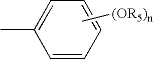

wherein n is 0, 1, 2 or 3;

R₅ is lower alkyl; and

R₆ is OH, lower alkyl, phenyl, benzyl or substituted phenyl, wherein the substituents are 1-3 groups independently selected from the group consisting of lower alkyl, lower alkoxy, carboxy, NO₂, NH₂, OH, halogeno, lower alkylamino and di-lower alkylamino;

(f) a sterol absorption inhibitor represented by Formula (VI):

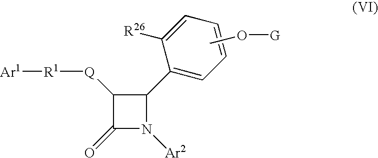

or isomers thereof, or pharmaceutically acceptable salts or solvates of the compounds of Formula (VI) or of the isomers thereof, wherein in Formula (VI):

R²⁶ is H or OG¹;

G and G′ are independently selected from the group consisting of H,

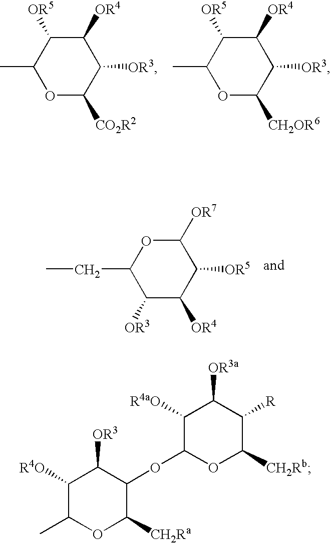

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy and —W—R³⁰;

wherein W is independently selected from the group consisting of —NH—C(O), —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

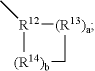

and

R¹ is selected from the group consisting of:

—(CH₂)_{q}—, wherein q is 2-6, provided that when Q forms a spiro ring, q can also be zero or 1;

—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;

—(C₂-C₆)alkenylene-; and

—(CH₂)_{f}—V—(CH₂)_{g}—, wherein V is C₃-C₆ cycloalkylene, f is 1-5 and g is 0-5, provided that the sum of f and g is 1-6;

R¹² is

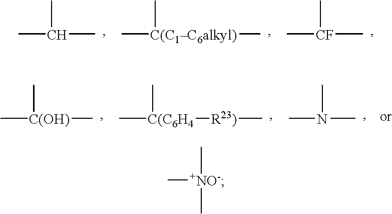

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)-group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, the R¹³'s can be the same or different; and

provided that when b is 2 or 3, the R¹⁴'s can be the same or different;

and when Q is a bond, R¹ also can be:

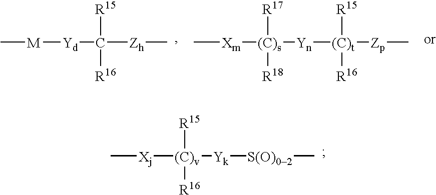

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R², —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R¹⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5; and when Q is a bond and R¹ is

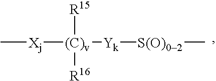

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy;

(g) a sterol absorption inhibitor represented by Formula (VII):

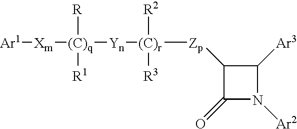

(VII)

or isomers thereof, or pharmaceutically acceptable salts or solvates of the compounds of Formula (VII) or of the isomers thereof, wherein in Formula (VII):

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(di-lower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1;

r is 0 or 1;

m, n and p are independently 0, 1, 2, 3 or 4;

provided that at least one of q and r is 1, and the sum of m, n, p, q and r is 1, 2, 3, 4, 5 or 6; and

provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₁₀OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷—NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl; and

(h) a sterol absorption inhibitor represented by Formula (IX):

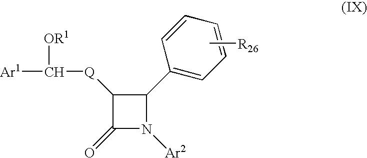

or isomers thereof, or pharmaceutically acceptable salts or solvates of the compounds of Formula (IX) or of the isomers thereof, wherein in Formula (IX):

R²⁶ is selected from the group consisting of:

a) OH;

b) OCH₃;

c) fluorine and

d) chlorine.

R¹ is selected from the group consisting of

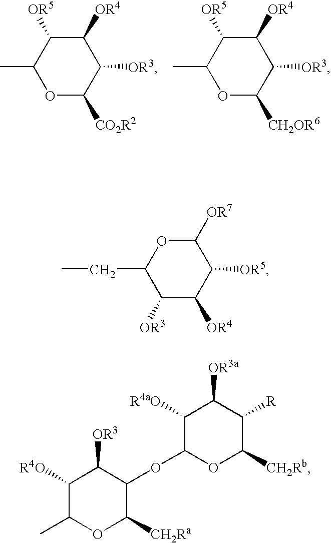

—SO₃H; natural and unnatural amino acids.

R, R^{a} and R^{b} are independently selected from the group consisting of H, —OH, halogeno, —NH₂, azido, (C₁-C₆)alkoxy(C₁-C₆)-alkoxy and —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is —(CH₂)_{q}—, wherein q is 2-6, or, with the 3-position ring carbon of the azetidinone, forms the spiro group;

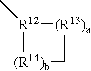

R¹² is

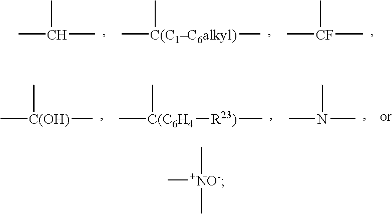

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)-group;

a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR⁹R²⁰, —(C₁-C₆ alkylene)-COOR¹⁹, —CH═CH—COOR¹⁹, —CF₃, —CN, —NO₂ and halogen;

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

²² is H, (C₁-C₆)alkyl, aryl (C₁-C₆)alkyl, —C(O)R¹⁹ or —COOR¹⁹;

²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

²⁵ is H, —OH or (C₁-C₆)alkoxy.

In another embodiment, the sterol absorption inhibitor is represented by Formula (VIII):

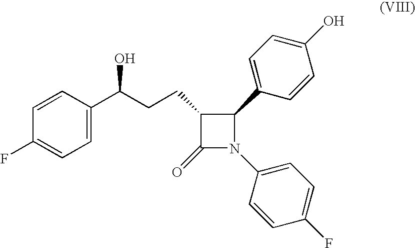

or pharmaceutically acceptable salts or solvates thereof.

In another embodiment, the sterol absorption inhibitor is ezetimibe.

Generally, the effective amount of sterol absorption inhibitor is that which is sufficient to provide a medically desirable result. The daily dose of the sterol absorption inhibitor(s) preferably ranges from about 0.1 to about 30 mg/kg of body weight per day, and more preferably about 0.1 to about 15 mg/kg. For an average body weight of 70 kg, the dosage level therefore ranges from about 1 mg to about 1000 mg of sterol absorption inhibitor(s) per day, given in a single dose or 2-4 divided doses. The exact dose, however, is determined by the attending clinician and is dependent on the potency of the compound administered, the age, weight, condition and response of the patient.

Compositions or formulations including sterol absorption inhibitors are disclosed in the references incorporated by reference above. An example of a suitable composition is:

Tablet A

| No. | Ingredient | mg/tablet |
|---|---|---|
| 1 | Ezetimibe | 10 |
| 2 | Lactose monohydrate NF | 55 |
| 3 | Microcrystalline cellulose NF | 20 |
| 4 | Povidone USP (K29-32) | 4 |
| 5 | Croscarmellose sodium NF | 8 |
| 6 | Sodium lauryl sulfate NF | 2 |
| 7 | Magnesium stearate NF | 1 |
| | Total | 100 |

A non-limiting example of a suitable formulation is ZETIA® ezetimibe formulation, which is commercially available from MSP Pharmaceuticals, Inc.

Furthermore, the invention provides for evaluating whether a subject may benefit from treatment with a sterol absorption inhibitor in combination with a statin, such as atorvastatin and simvastatin.

### EXAMPLES

### Example 1

### PROCAM Study

The relationship between modest elevations in phytosterols in the normal range and risk of coronary heart disease was investigated through a nested case-control study using samples from participants in the Prospective Cardiovascular Münster (PROCAM) study, a large-scale prospective epidemiological study of men and women at work in the northern German city of Münster and the adjoining region of the northern Ruhr valley.

Subjects and Methods

Recruitment to the PROCAM study was started in 1979 and completed in 1985. (Cullen P, Schulte H, Assmann G. The Münster Heart Study (PROCAM) Total mortality in middle-aged men is increased at low total and LDL cholesterol concentrations in smokers but not in nonsmokers. Circulation 1997;96:2128-36). During this time 20,060 employees of 52 companies and local government authorities were examined. At recruitment, participants were aged between 16 and 65 years. The baseline examination was performed by the same physician throughout the entire study period and included standardized questionnaires, measurement of blood pressure, body weight and height, a resting electrocardiogram (ECG), and a blood sample following a 12-hour fast for measurement of more than 20 laboratory parameters. Total serum cholesterol, HDL cholesterol, and triglycerides were measured using enzymatic and (for HDL cholesterol) a precipitation method from Boehringer Mannheim. LDL cholesterol was calculated by the Friedewald formula if triglycerides were 4.5 mmol/L (Friedewald W T, Levy J, Fredrickson D S. Estimation of the concentration of low-density-lipoprotein cholesterol in plasma, without use of the preparative ultracentrifuge. Clin. Chem. 1972;18:499-509). The examination was carried out free of charge during paid working hours. Participation was voluntary and ranged from 40% to 80% of eligible employees. All findings were reported to the participants' general practitioners, and the volunteers were informed if the results were normal or if a check-up by the general practitioner might be necessary. The investigators neither carried out nor arranged for any intervention.

Follow-up was by questionnaire every two years with a response rate of 96%. In each case in which evidence of morbidity or mortality was entered in the questionnaire, hospital records and records of the attending physician were obtained and, in the case of deceased study participants, an eyewitness account of death was sought. A coronary event was defined as the occurrence of sudden cardiac death or a definite fatal or nonfatal myocardial infarction on the basis of ECG and/or cardiac enzyme changes. Participants were excluded from follow-up if at the time of recruitment they had a history of either myocardial infarction or stroke or if the ECG at recruitment showed signs of ischaemic heart disease. Patients with a history of angina pectoris at recruitment, as defined using the Rose questionnaire (World Health Organization. Cardiovascular Survey Methods. Geneva: 1997), were excluded from the present analysis. At the time of the study, 10 years of follow-up and event adjudication had been completed.

In a nested case-control study, 160 men and 17 women who suffered a myocardial infarction or sudden coronary death (coronary event) within 10 years of follow-up in the Prospective Cardiovascular Münster (PROCAM) study were matched with 354 controls for sex, age, date of investigation and smoking status. Phytosterol concentrations were measured in stored serum samples using gas chromatography-mass spectrometry. Analysis was performed using conditional logistic regression.

In the present analysis, all covariates were based on baseline values. Hypertension was defined as treated hypertension and/or systolic blood pressure ≧140 and/or diastolic blood pressure ≧90 mmHg, diabetes mellitus was defined as known diabetes and/or fasting blood glucose ≧7.0 mmol/L, metabolic syndrome was defined as the presence of at least three of the following components: body mass index (BMI) >29.0 kg/m² in men or >27.5 kg/m² in women, triglycerides ≧1.72 mmol/L, HDL cholesterol <1.03 mmol/L in men or <1.29 mmol/L in women, blood pressure ≧130/85 mmHg, fasting blood glucose between 6.1 and 6.9 mmol/L. A positive family history was assumed if a first-degree relative of the participant had suffered a myocardial infarction below the age of 60 years.

Study Design

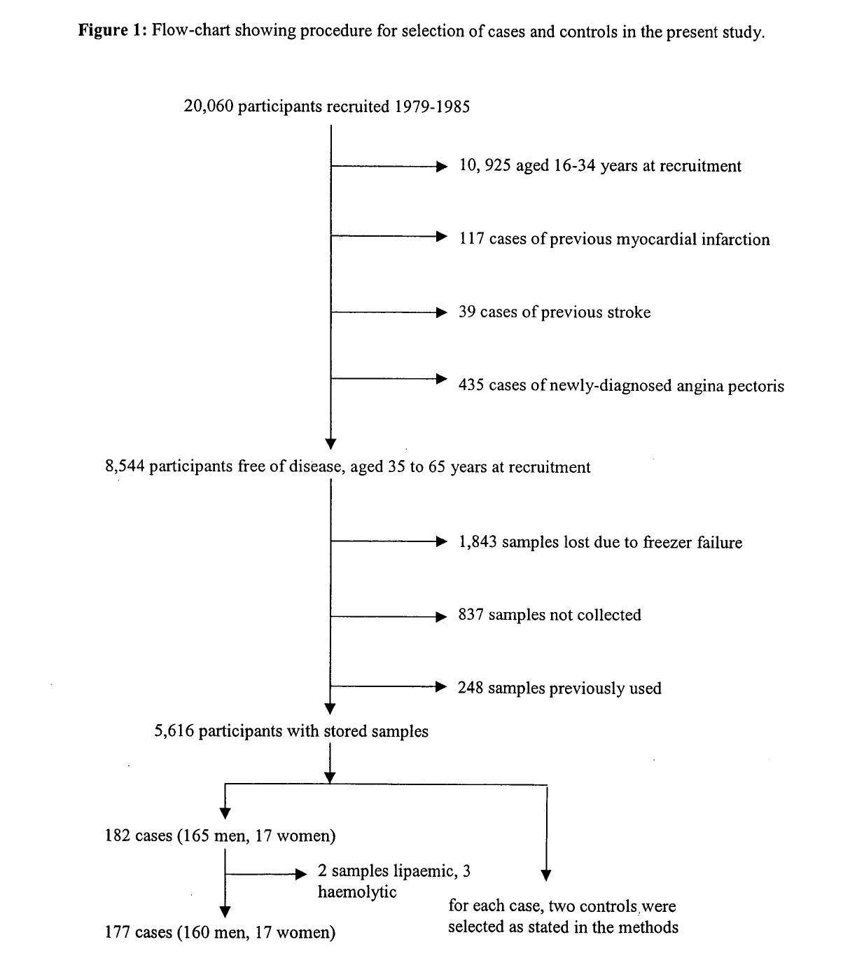

This was an observational nested case-control study of PROCAM participants aged 35 to 65 years at recruitment. This age cohort was chosen because it formed the basis of the PROCAM risk score (Assmann G, Cullen P, Schulte H. Simple scoring scheme for calculating the risk of acute coronary events based on the 10-year follow-up of the prospective cardiovascular Munster (PROCAM) study. Circulation 2002;105:310-5). Cases were defined as individuals who had suffered a coronary event during the 10-year follow-up period and who had stored plasma samples available for analysis. Two controls were chosen for each case from among the participants who were at risk of becoming a case at the time the case was diagnosed (risk set sampling). Controls were matched for year of entry into PROCAM, for sex and for baseline smoking status, and were required to have plasma samples available for analysis. Among the potential identified matches, the two closest in age to the case were selected. A flow chart of the selection procedure for participants in this study is shown in FIG. 1.

Analysis of Samples

Samples were removed from the freezer where they had been stored at −70° C. since collection and thawed at room temperature. Internal standards (10 μg epicoprostanol and 100 μg coprostanol) were added to 100 μL of plasma followed by saponification with 400 μL tetramethylammoniumhydroxide/isopropanol (20% w/v) for 15 min at 80° C. After cooling to room temperature, 800 μL of water were added and free cholesterol and non-cholesterol steroids were extracted with 200 μL of tetrachloroethylene (30 s vortex, 10 min centrifugation at 2000 g) (Klansek J J, Yancey P, St Clair R W, Fischer R T, Johnson W J, Glick J M. Cholesterol quantitation by GLC: artifactual formation of short-chain steryl esters. J. Lipid Res. 1995;36:2261-6). The organic fraction was evaporated and the residue containing the sterols was derivatized into trimethylsilylethers using 200 μL pyridine/hexamethyldisilazane/trimethylchlorosilane (9:3:1 v/v/v) at 60° C. for 30 min. After removal of excess reagents, the residue was dissolved in 100 μL n-hexane/pyridine (99:1 v/v), sonicated, and centrifuged at 2000 g for 5 min. The supernatant was analyzed by gas chromatography/mass spectrometry using a Finnigan GCQ equipped with an ion trap mass analyser and an SGE-HT5 fused silica capillary column as previously described (Kannenberg F. Entwicklung eines neuen Verfahrens zur Analyse von Gallensaturen mit Hilfe von Gaschromatographie/Massenspektrometrie-Kopplung. Identifizierung und Quantifizierung abnormer Metaboliten in Galle und Serum bei Storungen des Gallensäurenstoffwechsels. [dissertation]. 2000) for the phytosterols sitosterol, campesterol, cholestanol and lathosterol.

Data Analysis

The mean baseline values for continuous variables and the proportions for discrete risk factors in the case and control groups were calculated. To estimate the relative risk associated with elevated phytosterol concentrations or ratios, or with conventional risk factors, logistic regression analysis conditioned on the matching variables was applied. Conditional logistic regression was carried out using SPSS COXREG procedure. For continuous variables, the univariate relationship of each variable with risk of coronary events was analyzed. Each variable was split into 2 or 3 categories based on standard cut-points or clinical judgment and the univariate analyses using the categorical variables was repeated. The interaction between pairs of categorical variables by adding interaction term(s) in the model was also evaluated.

Results

Selection of Study Samples

As shown in FIG. 1, of the 20,060 participants in PROCAM recruited between 1979 and 1985, 11,516 were excluded because they were too young at recruitment, or because of previous myocardial infarction or stroke, or angina pectoris. Of the remaining 8,544 participants free of disease at recruitment, 2,928 were excluded because of freezer failure, or because samples had not been collected or used up in previous studies. Thus, 5,616 participants were eligible for inclusion. Of these 182 suffered a coronary event within 10 years of follow-up, of whom full analysis could be performed on 177 samples (5 cases were excluded from analysis because their blood samples were lipaemic or haemolytic). At baseline, one case each was taking bezafibrate, clofibrate and probucol; two controls were taking clofibrate and one control was taking fenofibrate.

Baseline Characteristics

In the present study, cases and controls were matched for sex, age, and smoking status. On univariate analysis, total and LDL cholesterol and triglycerides were greater among cases than among controls, while HDL cholesterol was lower. Cases also had higher systolic blood pressures. The metabolic syndrome and family history of myocardial infarction were also more common in the cases than in the controls (Table 1).

TABLE 1
Baseline characteristics of cases and controls in the present study.
All values expressed as mean ± standard deviation.
N.S. not significant.

| | Cases | Controls | |
|---|---|---|---|
| | (n = 177) | (n = 354) | P |
| Age (y) | 52.7 ± 6.9 | 52.7 ± 6.8 | — |
| Total cholesterol enz. (mmol/L) | 6.6 ± 1.2 | 6.0 ± 1.0 | 0.001 |
| LDL cholesterol (mmol/L) | 4.7 ± 1.2 | 4.0 ± 1.0 | <0.001 |
| HDL cholesterol (mmol/L) | 1.1 ± 0.3 | 1.2 ± 0.3 | 0.004 |
| Triglycerides (mmol/L) | 1.88 ± 1.15 | 1.61 ± 0.84 | 0.002 |
| Systolic blood pressure (mmHg) | 137 ± 20 | 131 ± 19 | 0.003 |
| Diastolic blood pressure (mmHg) | 86 ± 12 | 84 ± 11 | N.S. |
| Fasting blood glucose (mmol/L)) | 5.9 ± 1.3 | 5.7 ± 1.0 | 0.030 |
| Body mass index (kg/m²) | 26.8 ± 2.9 | 26.5 ± 3.2 | N.S. |
| Metabolic syndrome (%) | 29.7 | 21.5 | 0.044 |
| Family history of myocardial | 23.6 | 16.3 | 0.040 |
| infarction (%) | | | |
| Diabetes mellitus (%) | 11.3 | 7.1 | N.S. |
| Current smoker (%) | 44.6 | 44.6 | — |
| Former smoker (%) | 26.0 | 26.0 | — |
| Mean 10-year risk of myocardial | 20.3 ± 18.5 | 11.9 ± 12.0 | <0.001 |
| infarction* | | | |
| *In Men (160 cases and 320 controls) as estimated by the PROCAM risk score based on the Cox logistic regression model (Assmann G, Cullen P, Schulte H. Simple scoring scheme for calculating the risk of acute coronary events based on the 10-year follow-up of the prospective cardiovascular Munster | | | |
| # (PROCAM) study. Circulation 2002; 105: 310-5). | | | |

Phytosterol Levels

Of the four phytosterols measured, only concentrations of sitosterol and campesterol were higher in cases than in controls (Table 2). A parameter that has been used in previous studies is the ratio of phytosterol to total cholesterol (Rajaratnam R A, Gylling H, Miettinen T A. Independent association of serum squalene and noncholesterol sterols with coronary artery disease in postmenopausal women. J. Am. Coll. Cardiol. 2000;35:1185-91; Matthan N R, Giovanni A, Schaefer E J, Brown B G, Lichtenstein A H. Impact of simvastatin and niacin with and without antioxidants on plasma cholesterol absorption and synthesis markers in coronary artery disease patients with low HDL. J. Lipid Res. 2003). In the present study, none of the phytosterol/cholesterol ratios showed a statistically significant relationship with the occurrence of coronary events, however it is believed that this ratio is not indicative of the relationship between LDL and phytosterol levels (Table 2).

TABLE 2
Phytosterol concentrations and phytosterol/cholesterol ratios*
of cases and controls in the present study.
N.S. not significant.

| | | | | Molar phytosterol/cholesterol ratio | | |
|---|---|---|---|---|---|---|
| | Concentration (μmol/L) | | | (×10⁴) | | |
| | Cases | Controls | | Cases | Controls | |
| | (n = 177) | (n = 354) | P | (n = 177) | (n = 354) | P |
| Sitosterol | 5.03 ± 3.44 | 4.31 ± 2.38 | 0.003 | 8.82 ± 5.32 | 8.55 ± 4.33 | 0.489 |
| Campesterol | 10.39 ± 7.36 | 8.98 ± 5.26 | 0.010 | 18.02 ± 10.40 | 17.88 ± 9.91 | 0.868 |
| Cholestanol | 9.40 ± 4.65 | 8.77 ± 4.10 | 0.084 | 16.64 ± 6.19 | 17.52 ± 7.85 | 0.140 |
| Lathosterol | 4.50 ± 2.73 | 4.22 ± 2.42 | 0.233 | 8.05 ± 4.19 | 8.37 ± 4.43 | 0.407 |

Cholesterol measured by gas chromatography. To convert the phytosterol levels to μg/dL multiply as follows: sitosterol×41.5, campesterol×40.0, cholestanol×38.9, lathosterol×38.7. To convert cholesterol in mmol/L to mg/dL, multiply by 38.7.

Sitosterol and Coronary Risk

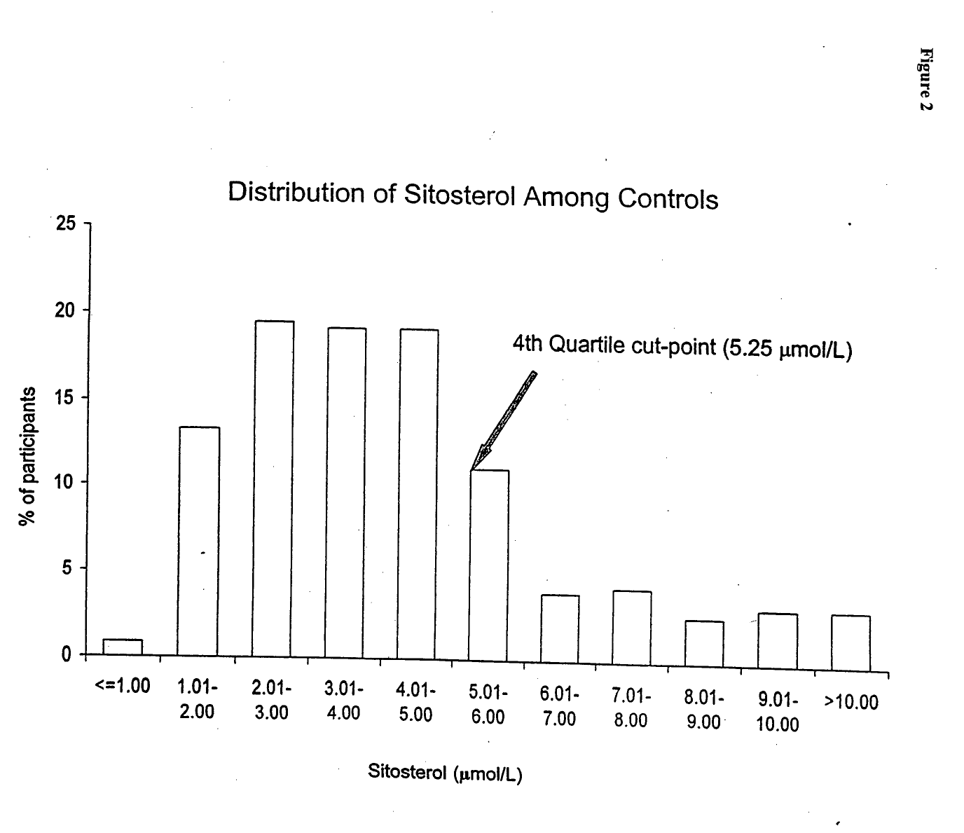

The distribution of sitosterol levels among controls was analyzed. A left-skewed distribution was observed with a peak between 2.01 and 5.00 μmol/L (FIG. 2). The level of 5.25 μmol/L was identified as the cut-off between the third and fourth quartiles, and levels above this were defined as high. The arrow shows the cut-off point of used to define the border between high and non-high levels.

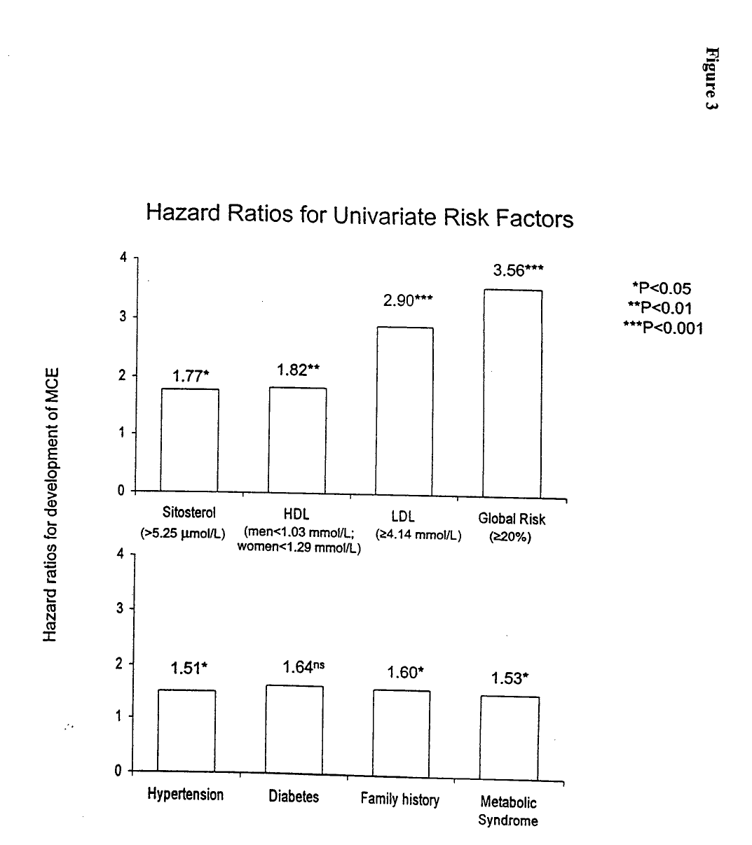

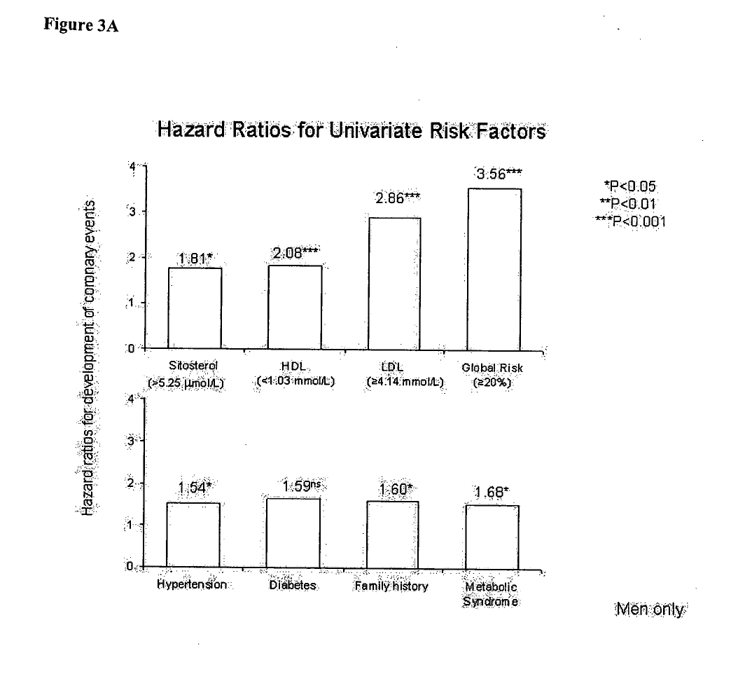

Next, the univariate hazard ratios for development of a coronary event for a number of established risk factors and for sitosterol in our population was calculated (FIG. 3) (FIG. 3A—men only). Referring to FIG. 3A, a high sitosterol (>5.25 μmol/L) level was associated with a risk (hazard ratio 1.81, p<0.05) similar to that of hypertension (1.54), family history (1.60) or the metabolic syndrome (1.68). Participants with diabetes mellitus did not have a significantly increased risk of coronary events compared to non-diabetic participants. In fact, of the univariate risk factors, only high LDL cholesterol (defined as ≧4.14 mmol/L, hazard ratio 2.86) and low HDL cholesterol (defined as <1.03 mmol/L in men, hazard ratio 2.08) was associated with greater risk of a coronary event than sitosterol (Executive Summary of the Third Report of the National Cholesterol Education Program (NCEP) Expert Panel on Detection, Evaluation, and Treatment of High Blood Cholesterol in Adults (Adult Treatment Panel III). JAMA 2001;285:2486-97). Not surprisingly, the greatest risk (3.56) was associated with the presence of a global risk of ≧20% in 10 years as calculated using the PROCAM risk score (Assmann G, Cullen P, Schulte H. Simple scoring scheme for calculating the risk of acute coronary events based on the 10-year follow-up of the prospective cardiovascular Munster (PROCAM) study. Circulation 2002; 105:310-5), which takes into account the eight variables of age, smoking status, systolic blood pressure, presence of diabetes, family history of myocardial infarction, LDL cholesterol, HDL cholesterol, and fasting triglyceride level.

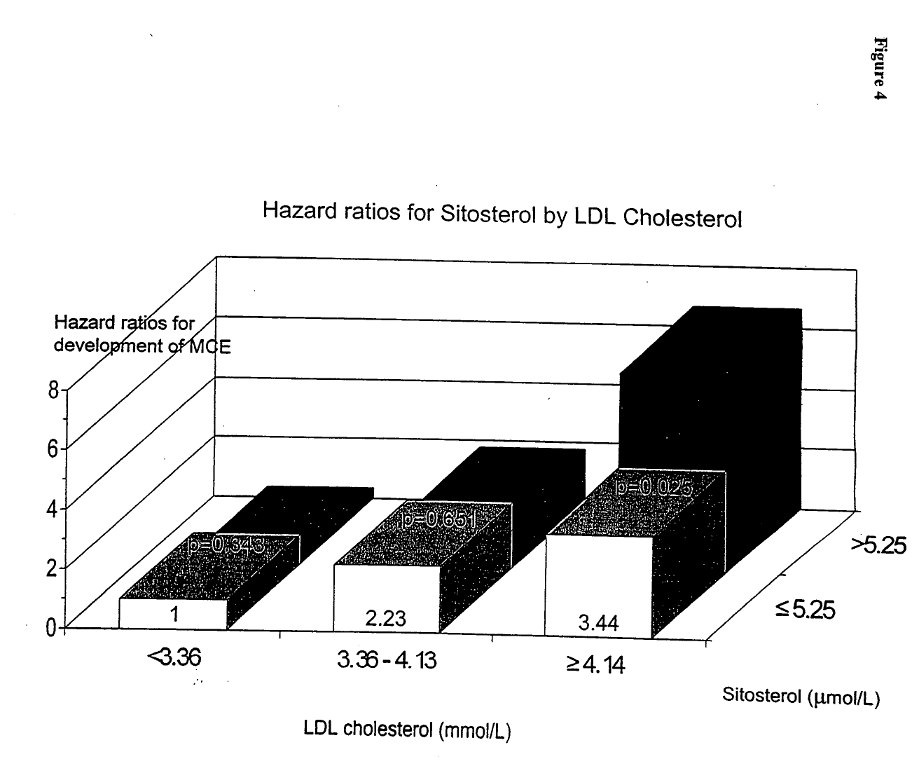

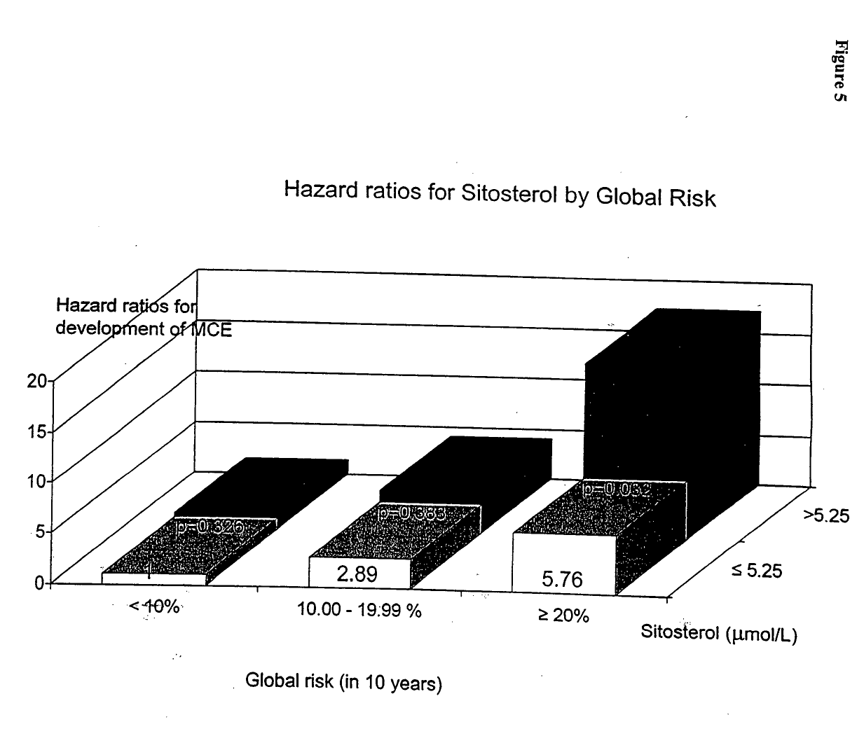

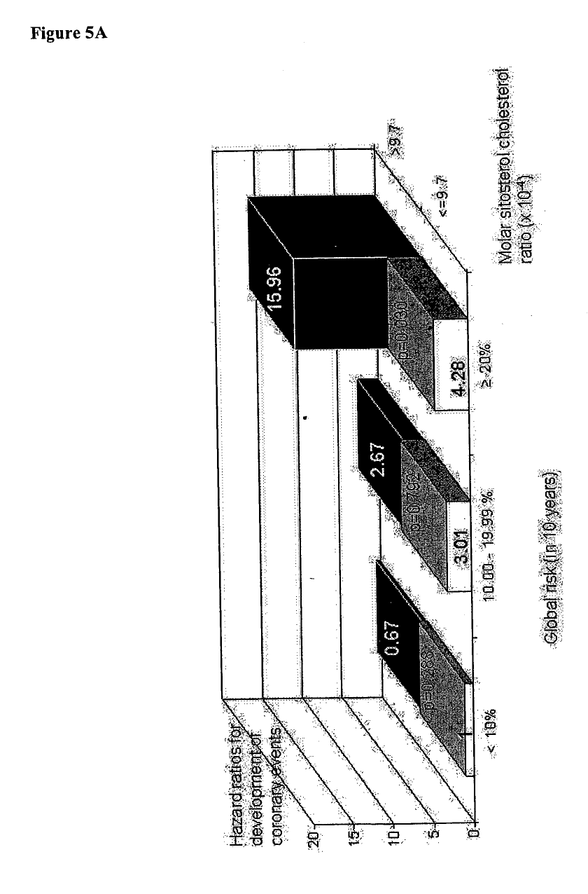

Finally, the interaction between sitosterol, LDL cholesterol, and global risk as estimated using the PROCAM risk score was examined (Assmann G, Cullen P, Schulte H. Simple scoring scheme for calculating the risk of acute coronary events based on the 10-year follow-up of the prospective cardiovascular Munster (PROCAM) study. Circulation 2002;105:310-5) (FIGS. 4 and 5). At high levels of LDL cholesterol (>4.13 mmol/L), high levels of sitosterol significantly increased risk of coronary events with an approximate doubling of the hazard ratio from 3.44 to 6.65 (p=0.025). At intermediate (3.36-4.13 mmol/L) and low (<3.36 mmol/L) levels of LDL cholesterol, the risk of coronary events was not increased at high sitosterol concentrations (FIG. 4). At high levels of global risk (≧20% risk of a coronary event in 10 years), further stratification by sitosterol level provided significant risk information, resulting in a three-fold increase in the hazard ratio for the participants with high levels of both, compared to the hazard ratio estimated for participants with high global risk alone (from 5.76 to 17.23, p=0.032). At intermediate (10.0%-<20% risk of a coronary event in 10 years) and lower (<10% risk in 10 years) levels of global risk, however, high sitosterol did not significantly increase the hazard ratio for coronary heart disease (FIG. 5). The hazard ratio for sitosterol did not change significantly after adjustment for other components of the PROCAM risk score. Similar interaction was observed for sitosterol to cholesterol ratio and global risk (FIG. 5A).

These results suggest that elevated sitosterol is associated with an increased risk for development of coronary events. Persons with a high LDL cholesterol or a high global risk of coronary heart disease who also had a high sitosterol concentration were two to three times more likely to develop a coronary event than those with lower levels of sitosterol.

The most important difference between this study and previous investigations is that phytosterol concentrations were measured using blood samples obtained prospectively, in most cases many years before the coronary event occurred. Since cases and controls were matched for year of entry, they were also effectively matched for plasma storage time. This means that the results are unlikely to be an artifact of patient selection or study design. The previous studies in this field were cross-sectional, while this study used a nested case-control design with risk set sampling. The latter design provided an estimation of risk that closely approximates the relative risk estimate that would have been obtained from a true prospective cohort study. Also, this study began before statins were available. Since few participants were receiving other lipid-modifying medication at the time their blood samples were drawn, it is unlikely that the results were substantially influenced by lipid-lowering therapy.

Of particular interest is the observation that increased sitosterol levels were only associated with increased risk for coronary heart disease in persons who were already at increased risk because of elevated LDL cholesterol or high global risk. It is possible that this relationship is merely a reflection of limited power to detect an effect in lower risk subgroups. However, it is analogous to an observation made in the case of lipoprotein (a) [Lp(a)]. Raised levels of Lp(a) appear to be associated with an increased atherosclerotic risk primarily in persons who also have high LDL cholesterol concentrations (Luc G, Bard J M, Arveiler D, Ferrieres J, Evans A, Amouyel P, Fruchart J C, Ducimetiere P. Lipoprotein (a) as a predictor of coronary heart disease: the PRIME Study. Atherosclerosis 2002;163:377-84.; von Eckardstein A, Schulte H, Cullen P, Assmann G. Lipoprotein(a) further increases the risk of coronary events in men with high global cardiovascular risk. J. Am. Coll. Cardiol. 2001;37:434-9.). The reason for the interaction between sitosterol and high LDL cholesterol in our population is not known, and this interaction remains to be confirmed in larger studies with greater power to detect potential associations in lower-risk subjects. It is also possible that sitosterol at high concentrations than reported here may be associated with increased coronary risk even among persons with normal LDL cholesterol.

This study cannot exclude the possibility that sitosterol itself is not the cause of increased coronary risk, but rather a surrogate for some other factor that impacts on the atherogenic process. However, elevated phytosterol levels are associated with severe premature atherosclerosis in homozygous sitosterolaemia, despite the fact that LDL cholesterol levels are generally normal or only modestly elevated in this condition (Björkhem I, Boberg K M. Inborn errors in bile acid biosynthesis and storage of sterols other than cholesterol. In: Scriver C R, Beaudet A L, Sly W S, Valle D, editors. The Metabolic and Molecular Bases of Inherited Disease on CD-ROM. 7 ed. New York: McGraw-Hill; 1997). In addition, since phytosterols are found within atherosclerotic lesions in these patients, it is possible that they are directly atherogenic (Salen G, Horak I, Rothkopf M, Cohen J L, Speck J, Tint G S, Shore V, Dayal B, Chen T, Shefer S. Lethal atherosclerosis associated with abnormal plasma and tissue sterol composition in sitosterolemia with xanthomatosis. J. Lipid Res. 1985;26:1126-33). Clearly, further research is warranted to evaluate the potential biochemical mechanisms. Further studies on the metabolism of sitosterol at the whole body and cellular level, as well as studies of the potential atherogenicity of lipoprotein-associated phytosterols, will be required to clarify this issue.

It is noteworthy that different lipid-lowering agents have different effects on plasma phytosterol concentrations. Statins have been reported to increase ratios of sitosterol and campesterol to cholesterol, and in some studies also increase absolute phytosterol concentrations (Miettinen T A, Gylling H, Lindbohm N, Miettinen T E, Rajaratnam R A, Relas H. Serum noncholesterol sterols during inhibition of cholesterol synthesis by statins. J. Lab Clin. Med. 2003;141:131-7). In contrast, bile acid sequestrants (Salen G, Horak I, Rothkopf M, Cohen J L, Speck J, Tint G S, Shore V, Dayal B, Chen T, Shefer S. Lethal atherosclerosis associated with abnormal plasma and tissue sterol composition in sitosterolemia with xanthomatosis. J. Lipid Res. 1985;26:1126-33) and cholesterol absorption inhibitors (Sudhop T, Gottwald B M, von Bergmann K. Serum plant sterols as a potential risk factor for coronary heart disease. Metabolism 2002;51:1519-21) have been shown to reduce phytosterol concentrations.

Elevated levels of sitosterol were associated with an increased risk of coronary events in patients with elevated LDL cholesterol levels or at high global risk. Even modest elevations in sitosterol are associated with the increased risk of coronary events, particularly in persons with high LDL cholesterol or high global risk of coronary heart disease. Such elevated levels may be used for identifying patients who may benefit from appropriate lipid-lowering therapy.

In summary, sitosterol levels were elevated in coronary event cases compared to controls (5.03±3.44 μmol/L [mean±SD] vs. 4.31±2.38 μmol/L respectively, p=0.003) (4.94±3.44 μmol/L vs. 4.27±2.38 μmol/L, p=0.028 (men only)). The upper quartile of sitosterol (>5.25 μmol/L) was associated with a 1.8-fold increase in risk (95% CI 1.1-2.8, p=0.014) (hazard ratio 1.81, p<0.05 (men only)) compared to the lower three quartiles. Persons with both high low density lipoprotein (LDL) cholesterol (>4.14 mmol/L) and high sitosterol (>5.25 μmol/L) were at a 1.9-fold (p=0.025) increased risk compared to those with high LDL cholesterol and low sitosterol (≦5.25 μmol/L). Among men with a global coronary event risk of >20% in 10 years as calculated using the PROCAM algorithm, high sitosterol levels were associated with an additional 3-fold increase in risk (p=0.032).

### Example 2

### Effects of Ezetimibe (EZE) and Simvastatin Administration to Hypercholesteremia Patients

The cholesterol absorption inhibitor, ezetimibe (EZE), blocks the intestinal absorption of cholesterol and phytosterols in both sitosterolemic and hypercholesterolemic patients. (Salen et al. Circulation 2004; 109:966-971; Sudhop et al. Circulation 2002; 106:1943-1948.)

Statins decrease cholesterol biosynthesis and appear to increase (atorvastatin) or have no effect (simvastatin) on phytosterols. (Miettinen et al. J Lab Clin Med 2003; 141:131-137).

Coadministered EZE and statin produces significant incremental reductions in low-density lipoprotein cholesterol (LDL-C) beyond that of statin alone. (Davidson et al. J Am Coll Cardiol 2002; 40:2125-2134; Ballantyne et al. Circulation 2003; 107:2409-2415.) However, the effect of EZE+statin on phytosterols is unknown. The present example evaluates the effects of EZE and simvastatin, or Zocor®, (SIMVA) on cholesterol synthesis and phytosterol absorption in hypercholesteremia patients. Specifically, the effects of EZE, simvastatin (SIMVA), and EZE/SIMVA coadministration on phytosterols (sitosterol and campesterol) and cholesterol precursors (lathosterol and desmosterol) in patients with primary hypercholesterolemia were evaluated.

Patients and Study Design

A post-hoc analysis of a randomized, double-blind, placebo-controlled trial of primary hypercholesterolemia patients (LDL-C 145-250 mg/dl and TG≦350 mg/dl) was conducted to examine the effects of 12 weeks of daily treatment with ezetimibe (EZE) 10 mg, simvastatin (SIMVA) pooled doses 10-80 mg, or EZE 10 mg/SIMVA 10-80 mg (EZE/SIMVA) coadministration (pooled) on concentrations of phytosterols (sitosterol and campesterol) and cholesterol precursors (lathosterol and desmosterol). Baseline and endpoint plasma samples of 578 patients were analyzed by GC-mass spectrometry

Following a 4 week placebo lead-in/diet phase, patients with primary hypercholesterolemia (LDL-C 145-250 mg/dL and TG≦350 mg/dL) were randomized on one of the following daily treatments for 12 weeks:

Placebo (PBO)

EZE 10 mg (ezetimibe formulation (see Tablet A above))

SIMVA monotherapy (10, 20, 40, or 80 mg) (ZOCOR® simvastatin commercially available from Merck & Co., Inc. encapsulated in No. 000 blue, opaque gelatin capsules)

EZE/SIMVA (10/10, 10/20, 10/40 or 10/80 mg) (coadministration of one tablet of ezetimibe formulation (see Tablet A above) and one tablet of ZOCOR® simvastatin commercially available from Merck & Co., Inc. encapsulated in No. 000 blue, opaque gelatin capsules)

Non-cholesterol Sterol Analysis

Of 668 patients randomized to treatment in the original study, 578 (86%) had evaluable −70° C. plasma samples for both baseline and endpoint visits. Concentrations of non-cholesterol sterols were measured by gas chromatography-mass spectrometry (GC-MS).

The primary efficacy parameter was mean percent change in plasma sitosterol concentration from baseline to endpoint comparing EZE/SIMVA (pooled across doses) versus SIMVA monotherapy (pooled across doses). Other efficacy parameters included mean percent changes in campesterol, lathosterol and desmosterol concentrations as well as mean changes in the ratios of each of these sterols to cholesterol.

Two-tailed testing was conducted at a=0.05. To compare treatment effects on sterol concentrations and ratios, an ANCOVA model including the fixed effects of treatment, dose, and dose-by-treatment interaction was used with the baseline value as the covariate.

TABLE 3
Baseline Patient Characteristics and Sterol Concentrations

| | Placebo | EZE 10 mg | SIMVA | EZE/SIMVA |
|---|---|---|---|---|
| Characteristic | (n = 62) | (n = 55) | (n = 232) | (n = 229) |
| Age, yr | 59.0 (12.5) | 59.6 (11.1) | 56.8 (12.1) | 57.5 (12.0) |
| Female gender, No. (%) | 35 (56) | 32 (58) | 136 (59) | 128 (56) |
| White, No. (%) | 59 (95) | 53 (96) | 210 (90) | 212 (93) |
| Body mass index, kg/m²* | 28.9 (4.8) | 28.6 (5.0) | 28.9 (5.0) | 29.0 (5.1) |
| LDL cholesterol, mg/dL | 176.8 (20.4) | 181.2 (23.5) | 178.7 (20.5) | 175.6 (19.1) |
| HDL cholesterol, mg/dL | 53.2 (12.2) | 50.5 (10.9) | 51.0 (10.4) | 50.3 (11.9) |
| Total cholesterol, mg/dL | 264.6 (23.6) | 271.2 (28.8) | 264.7 (25.0) | 263.6 (25.0) |
| Triglycerides, mg/dL | 156.7 (85.0) | 182.7 (104.8) | 155.3 (78.1) | 172.3 (90.2) |
| Sitosterol, mg/dL | 0.24 (0.13) | 0.23 (0.15) | 0.21 (0.11) | 0.23 (0.15) |
| Campesterol, mg/dL | 0.44 (0.23) | 0.39 (0.24) | 0.38 (0.19) | 0.41 (0.26) |
| Lathosterol, mg/dL | 0.19 (0.10) | 0.20 (0.09) | 0.20 (0.12) | 0.20 (0.09) |
| Desmosterol, mg/dL | 0.15 (0.08) | 0.19 (0.11) | 0.14 (0.08) | 0.16 (0.08) |

Values are mean (SD) except where otherwise noted.
*Data for body mass index not available for one person in the placebo control group.

Demographics and baseline characteristics were generally well matched across treatment groups (Table 3).

Results

EZE significantly lowered concentrations of phytosterols and increased the cholesterol precursor-lathosterol. SIMVA significantly lowered concentrations of cholesterol precursors but did not affect phytosterol concentrations. EZE/SIMVA coadministration lowered both concentrations of phytosterols and cholesterol precursors. Results for the sterol:cholesterol ratios were similar to those for concentrations, except that SIMVA increased ratios of phytosterol:cholesterol.

TABLE 4

| | PBO | | EZE 10 mg | | SIMVA | | EZE/SIMVA | |
|---|---|---|---|---|---|---|---|---|
| | (n = 62) | | (n = 55) | | (n = 232) | | (n = 229) | |
| | % | | % | | % | | % | |
| Sterol | change | Ratio | change | Ratio | change | Ratio | change | Ratio |
| Sitosterol | −0.5 | −4.1 | −46.5* | −37.8* | −3.3* | 21.4* | −52.1*† | −25.1*† |
| Campesterol | −2.0 | −6.8 | −50.5* | −78.6* | −1.6* | 39.8* | −60.7*† | −68.3*† |
| Lathosterol | 4.7 | 2.7 | 35.5* | 36.8* | −53.5* | −37.8* | −47.6*† | −22.1*† |
| Desmosterol | 6.5 | 5.1 | 16.1 | 25.7* | −48.0* | −22.8* | −45.6*† | −11.1† |

SIMVA = pool of all doses of SIMVA.
Comparisons:
EZE & SIMVA vs PBO;
EZE/SIMVA vs SIMVA
% change:
Mean % change. Ratio: Mean change (10² mmol/mol)
*P < 0.001 vs PBO;
^{†}P < 0.001 vs SIMVA (pooled) P < 0.001 vs EZE

The net effect of EZE/SIMVA is to inhibit cholesterol synthesis and the absorption of phytosterols (in conjunction with cholesterol). Clinical relevance and implications with respect to atherosclerosis development and progression warrant further studies.

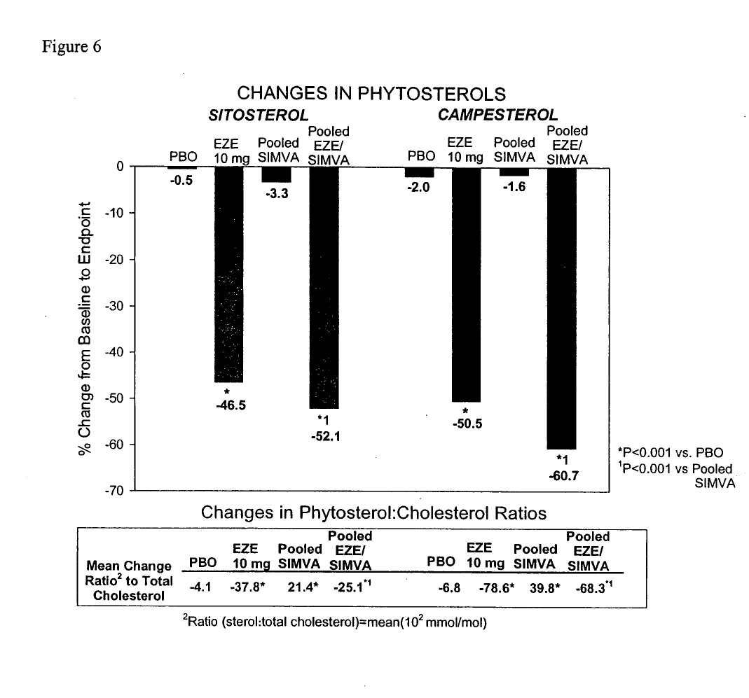

Treatment with EZE or pooled EZE/SIMVA produced large, significant reductions (from baseline to endpoint) in the concentrations of sitosterol and campesterol (p<0.001 for EZE vs. PBO; p<0.001 for EZE/SMIVA vs. PBO and vs. SIMVA monotherapy; FIG. 6). Pooled SIMVA monotherapy had no significant effect on phytosterol concentrations.

EZE and pooled EZE/SIMVA also significantly reduced the ratios of sitosterol:cholesterol and campesterol:cholesterol (p<0.001 for EZE vs. PBO; p<0.001 for EZE/SIMVA vs. PBO and vs. SIMVA monotherapy; Table in FIG. 6). These findings are consistent with the large effects seen on phytosterol concentrations. In contrast, SIMVA monotherapy significantly increased both ratios relative to PBO (p<0.001).

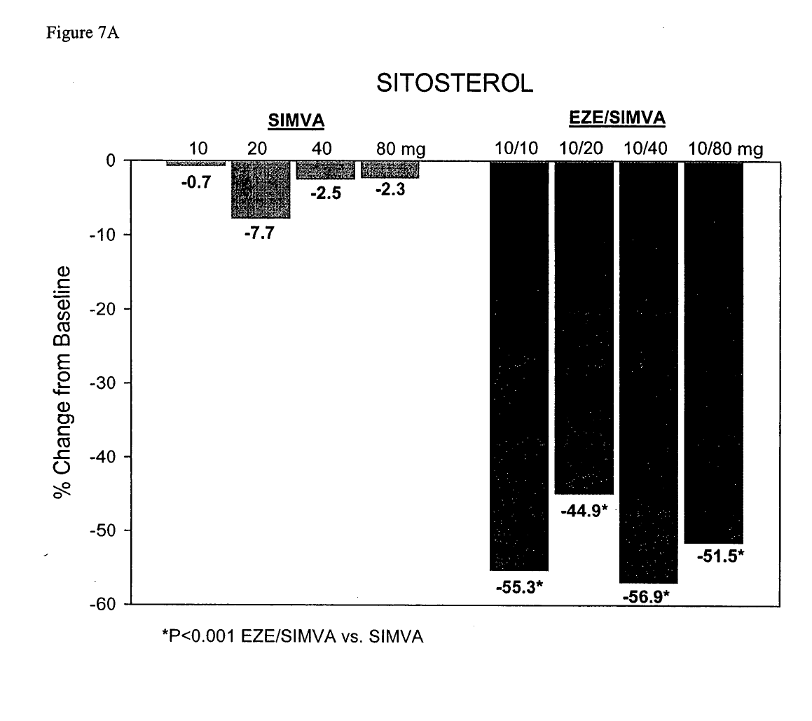

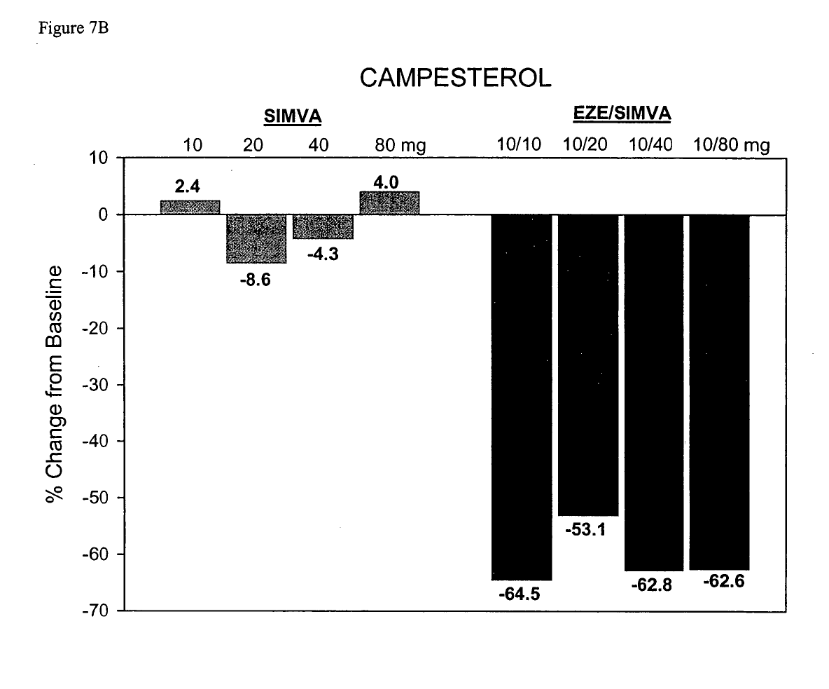

The effect of EZE/SIMVA on sitosterol and campesterol appeared to be independent of SIMVA dose (FIGS. 7A and 7B).

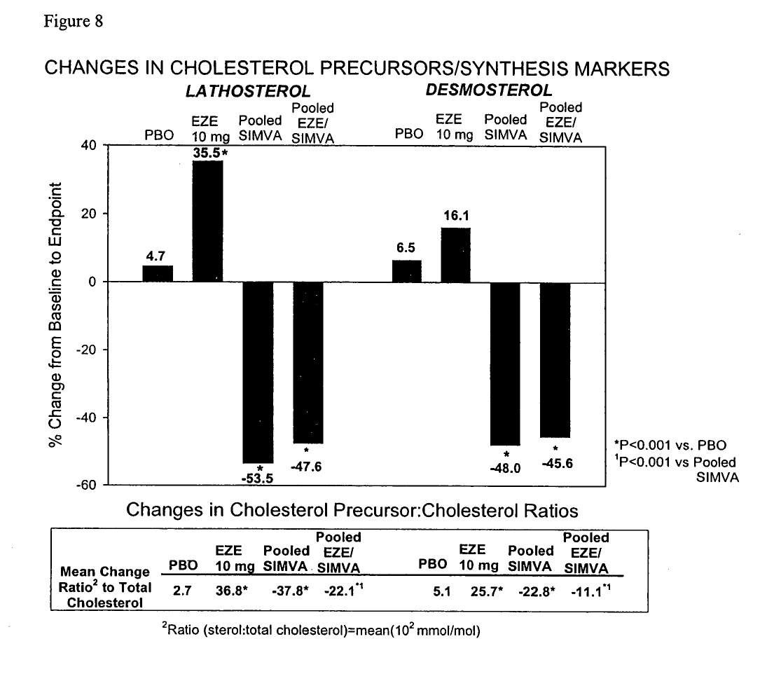

Pooled SIMVA monotherapy and pooled EZE/SIMVA produced large, significant reductions (from baseline to endpoint) in the concentrations of the cholesterol precursors, lathosterol and desmosterol (p<0.001 for SIMVA vs. PBO; p<0.001 for pooled EZE/SIMVA vs. PBO and vs. EZE; FIG. 8). EZE increased the concentrations of both precursors (p<0.001 vs. PBO for lathosterol; increases were not significant for desmosterol).

Pooled SIMVA monotherapy and pooled EZE/SIMVA also significantly reduced the ratios of lathosterol:cholesterol and desmosterol:cholesterol (p<0.001 for SIMVA vs. PBO; p<0.001 for EZE/SIMVA vs. PBO and vs. EZE; Table in FIG. 8). Both ratios were reduced more by SIMVA monotherapy than by the coadministration (p<0.001). EZE monotherapy significantly increased both ratios relative to placebo (p<0.001).

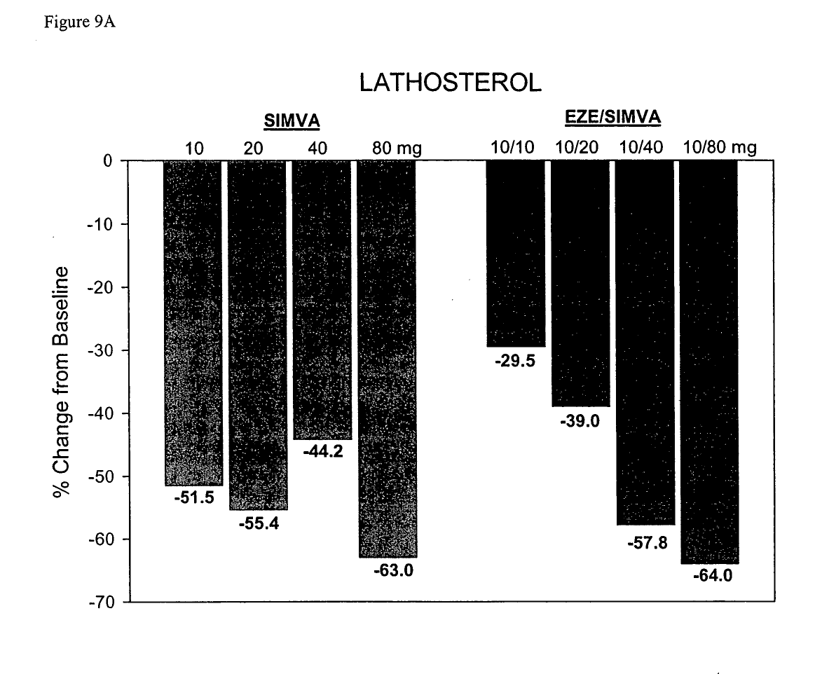

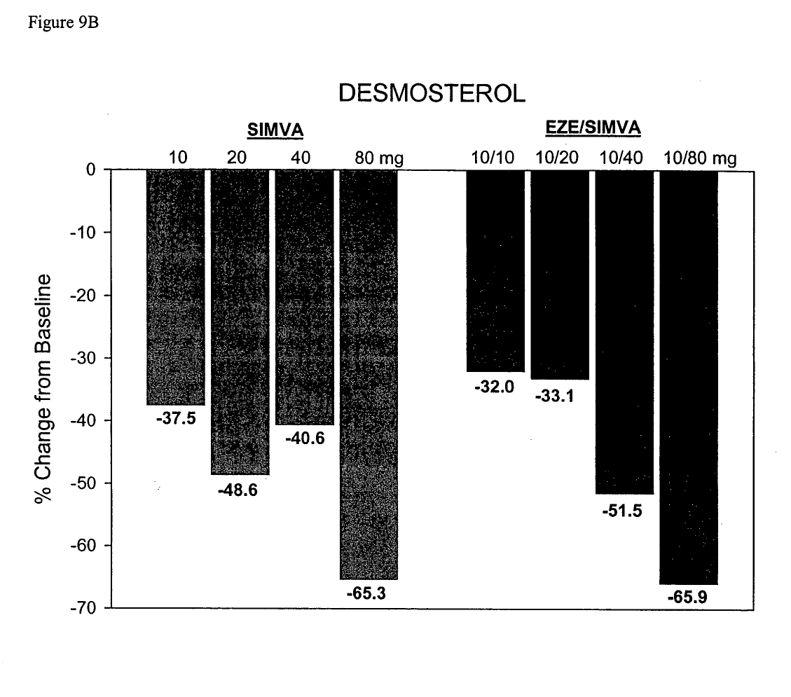

The effects of EZE/SIMVA on lathosterol and campesterol increased with increasing SIMVA dose (FIGS. 9A and 9B).

TABLE 5
Changes in Non-cholesterol Sterols in Relation to Changes
in LDL-C and Other Lipid Parameters by Treatment

| | LS Mean % Change (±SE)* from Baseline | | | |
|---|---|---|---|---|
| | | | | Pooled |
| | Placebo | EZE-10 mg | Pooled SIMVA | EZE/SIMVA |
| Parameter | (N = 62) | (N = 55) | (N = 232) | (N = 229) |
| LDL-C | −0.5 ± 1.7 | −18.5 ± 1.8 | −37.1 ± 0.9 | −51.4 ± 0.9¹ |
| Total | 0.4 ± 1.3 | −13.5 ± 1.4 | −26.8 ± 0.7 | −37.8 ± 0.7¹ |
| Cholesterol | | | | |
| HDL-C | 1.0 ± 1.5 | 5.5 ± 1.6 | 7.2 ± 0.8 | 9.8 ± 0.8^{‡} |
| Triglycerides* | 1.2 ± 4.0 | −8.6 ± 3.9 | −20.4 ± 1.5 | −30.2 ± 1.6¹ |
| (median) | | | | |

HDL-C = high-density lipoprotein cholesterol
*Median and SE are from raw data
¹P < 0.0001 vs Pooled SIMVA;
^{‡}P < 0.05 vs Pooled SIMVA
EZE = ezetimibe;
SIMVA = simvastatin;
LDL-C = low-density lipoprotein cholesterol

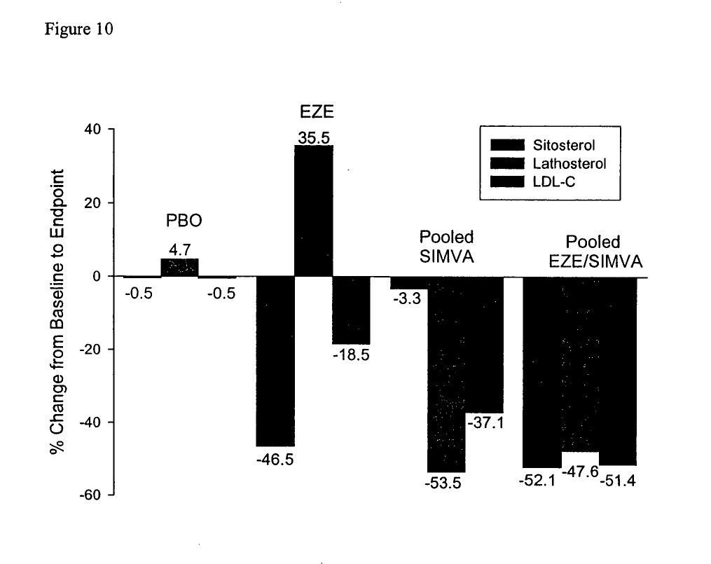

See FIG. 10.

Relative to SIMVA monotherapy, EZE/SIMVA significantly reduces LDL-C, total cholesterol, and triglycerides.

EZE, SIMVA monotherapy and EZE/SIMVA all produce significant changes in LDL-C, total cholesterol, and triglycerides relative to baseline.

EZE/SIMVA significantly reduces phytosterols and cholesterol precursors as well as LDL-C.

The results of this analysis demonstrated that, in patients with primary hypercholesterolemia EZE monotherapy significantly reduced plasma concentrations of the phytosterols, sitosterol and campesterol. EZE monotherapy increased plasma concentrations of the cholesterol precursors, lathosterol and desmosterol (markers of cholesterol synthesis.) SIMVA monotherapy had no effect on plasma phytosterol concentrations but significantly increased the ratios of sitosterol:cholesterol and campesterol:cholesterol most likely due to decreases in cholesterol concentrations, reflecting inhibition of cholesterol synthesis. EZE/SIMVA significantly reduced plasma concentrations of both phytosterols and sterol markers of cholesterol synthesis relative to PBO.

These findings are consistent with the known complementary mechanisms of action of EZE and SIMVA on sterol metabolism. EZE blocks the intestinal uptake of cholesterol and non-cholesterol sterols which in turn leads to reduced plasma concentrations and a compensatory increase in endogenous cholesterol synthesis. SIMVA inhibits cholesterol synthesis in the liver but has no effect on the concentrations of phytosterols (which are not synthesized endogenously.)

Thus, through dual inhibition of sterol absorption and cholesterol synthesis, EZE/SIMVA coadministration produces large, multitargeted reductions in cholesterol and non-cholesterol sterols in patients with primary hypercholesterolemia.

### Example 3

### Effect of Ezetimibe and Atorvastatin Adminstration in Hypercholesteremia Patients

The present example evaluates the effects of EZE and atorvastatin, or Lipitor®, (ATORVA) on cholesterol synthesis and phytosterol absorption in hypercholesteremia patients. Specifically, the effects of EZE, simvastatin (ATORVA), and EZE/ATORVA coadministration on phytosterols (sitosterol and campesterol) and cholesterol precursors (lathosterol and desmosterol) in patients with primary hypercholesterolemia were evaluated.

Patients and Study Design

A post-hoc analysis of a randomized, double-blind, placebo-controlled trial of primary hypercholesterolemia patients (LDL-C 145-250 mg/dl and TG<350 mg/dl) was conducted. Baseline and endpoint samples of 397 patients were analyzed by GCMS

In this trial, following a 4 week placebo lead-in/diet phase, patients with primary hypercholesterolemia (LDL-C 3.8-6.5 mmol/L [145-250 mg/dL] and TG≦4 mmol/L [350 mg/dL]) were randomized to one of the following daily treatments for 12 weeks:

Placebo (PBO)

ATORVA monotherapy (10, 20, 40, or 80 mg) (LIPITOR atorvastatin commercially available from Pfizer encapsulated in No. 0 blue, opaque gelatin capsules)

EZE 10 mg (ezetimibe formulation (see Tablet A above)EZE/ATORVA (10/10, 10/20, 10/40 or 10/80 mg) (coadministration of one tablet of ezetimibe formulation (see Tablet A above) and one tablet of LIPITOR® atorvastatin commercially available from Pfizer encapsulated in No. 0 blue, opaque gelatin capsules.

Non-Cholesterol Sterol Analysis

Of 628 patients randomized to treatment in the original study, 397 (63%) had evaluable −70° C. plasma samples for both baseline and endpoint visits.

Concentrations of non-cholesterol sterols were measured by gas chromatography-mass spectrometry (GC-MS).

The primary efficacy parameter was mean percent change in plasma sitosterol concentration from baseline to endpoint comparing EZE/ATORVA (pooled across doses) versus ATORVA (pooled across doses). Other efficacy parameters included mean percent changes in campesterol, lathosterol and desmosterol concentrations as well as mean changes in the ratios of each of these sterols to cholesterol.

Two-tailed testing was conducted at a=0.05. To compare treatment effects on sterol concentrations and ratios, an ANCOVA model including the fixed effects of treatment, dose, and dose-by-treatment interaction was used with the baseline value as the covariate.

TABLE 6
Baseline Patient Characteristics and Sterol Concentrations

| | Placebo | EZE 10 mg | ATORVA | EZE/ATORVA |
|---|---|---|---|---|
| Characteristic | (n = 41) | (n = 39) | (n = 160) | (n = 157) |
| Age, yr | 57.7 (11.6) | 56.8 (11.3) | 57.9 (11.5) | 59.5 (11.4) |
| Female gender, No. (%) | 20 (49) | 23 (59) | 101 (63) | 89 (57) |
| White, No. (%) | 33 (81) | 34 (87) | 130 (81) | 137 (87) |
| Body mass index, kg/m² | 27.2 (3.7) | 29.2 (9.0) | 28.4 (4.7) | 28.9 (5.0) |
| LDL cholesterol, mmol/L | 4.47 (0.54) | 4.46 (0.44) | 4.62 (0.54) | 4.58 (0.55) |
| HDL cholesterol, mmol/L | 1.32 (0.30) | 1.33 (0.33) | 1.39 (0.33) | 1.30 (0.32) |
| Total cholesterol, mmol/L | 6.65 (0.68) | 6.64 (0.50) | 6.95 (0.65) | 6.88 (0.72) |
| Triglycerides, | 1.51 (1.00) | 1.61 (0.62) | 1.84 (0.92) | 1.95 (0.95) |
| Sitosterol, mmol/L | 0.008 (0.004) | 0.008 (0.004) | 0.008 (0.005) | 0.007 (0.004) |
| Campesterol, mmol/L | 0.017 (0.010) | 0.016 (0.010) | 0.015 (0.010) | 0.014 (0.008) |
| Lathosterol, mmol/L | 0.006 (0.003) | 0.007 (0.003) | 0.007 (0.003) | 0.007 (0.004) |
| Desmosterol, mmol/L | 0.006 (0.003) | 0.005 (0.002) | 0.006 (0.002) | 0.009 (0.028) |

Values are mean (SD) except where otherwise noted.
Demographics and baseline characteristics were generally well matched across treatment groups (Table 6).

TABLE 7

| | LS Mean % Change (±SE)* from Baseline | | | |
|---|---|---|---|---|
| | | | Pooled | Pooled |
| | Placebo | EZE-10 mg | ATORVA | EZE/ATORVA |
| Parameter | (N = 40) | (N = 39) | (N = 160) | (N = 157) |
| LDL-C | 6.87 (2.01) | −17.10 (2.06) | −43.56 (1.02) | −56.40 (1.03)¹ |
| Total Cholesterol | 4.39 (1.59) | −12.97 (1.63) | −32.96 (0.81) | −42.88 (0.82)¹ |
| HDL-C | 5.18 (1.67) | 5.36 (1.71) | 5.21 (0.85) | 18.83 (0.86)^{‡} |
| Triglycerides* | −6.25 (6.65) | −2.86 (4.54) | −25.83 (1.80) | −35.94 (1.84)¹ |
| (median) | | | | |

*Median and SE are from raw data
¹P < 0.0001 vs. Pooled ATORVA;
^{‡}P < 0.005 vs. Pooled ATORVA
EZE = ezetimibe;
ATORVA = atorvastatin;
LDL-C = low-density lipoprotein cholesterol;
HDL-C = high-density lipoprotein cholesterol

Results

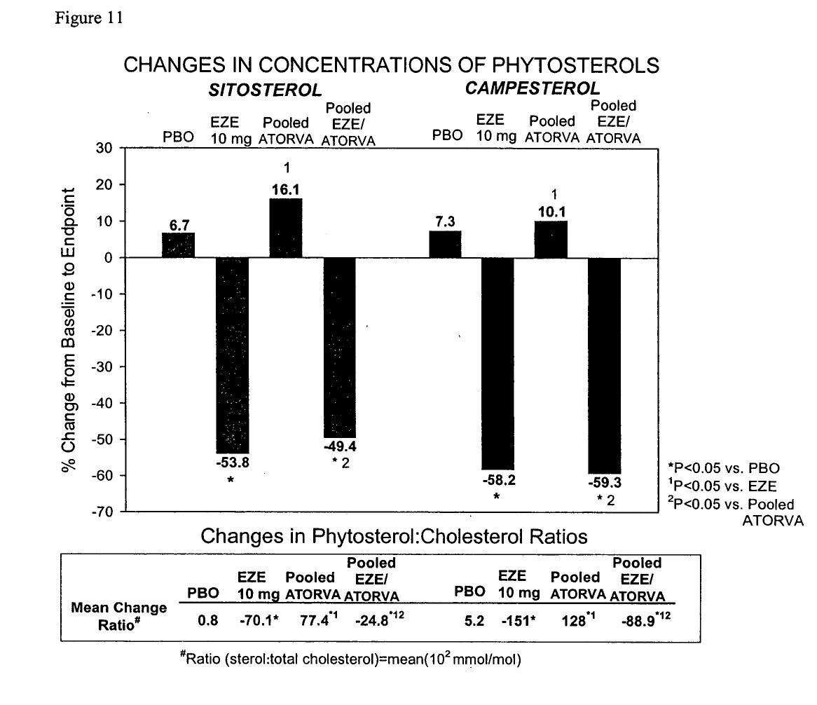

Treatment with EZE or pooled EZE/ATORVA produced large, significant reductions (from baseline to endpoint) in the concentrations of sitosterol and campesterol (p<0.05 for EZE vs. PBO; p<0.05 for EZE/ATORVA vs. PBO and vs. ATORVA; FIG. 11). Pooled ATORVA significantly increased phytosterol concentrations (p<0.05 vs. EZE).

EZE and pooled EZE/ATORVA also significantly reduced the ratios of sitosterol:cholesterol and campesterol:cholesterol (p<0.05 for EZE vs. PBO; p<0.05 for EZE/ATORVA vs. PBO, vs. ATORVA, and vs. EZE; Table in FIG. 11). These findings are consistent with the large effects on phytosterol concentrations. In contrast, ATORVA significantly increased both ratios (p<0.05) relative to PBO and to EZE.

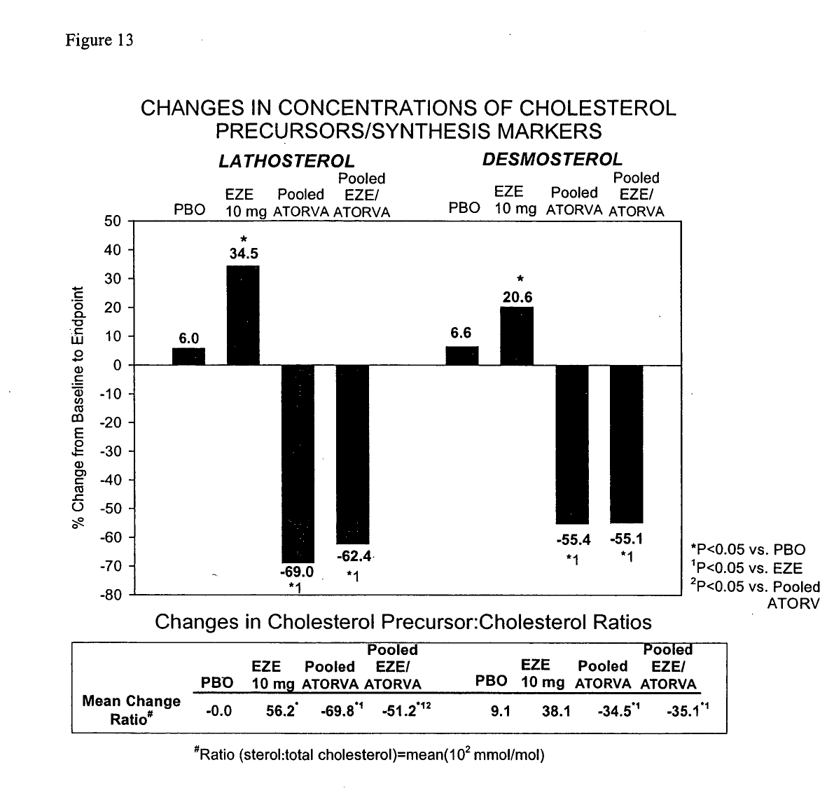

Pooled ATORVA and pooled EZE/ATORVA produced large, significant reductions (from baseline to endpoint) in the concentrations of the cholesterol precursors, lathosterol and desmosterol (p<0.05 for ATORVA vs. PBO and vs. EZE; p<0.05 for pooled EZE/ATORVA vs. PBO and vs. EZE; FIG. 13). EZE significantly increased the concentrations of both precursors relative to PBO (p<0.05).

Pooled ATORVA and pooled EZE/ATORVA also significantly reduced the ratios of lathosterol:cholesterol and desmosterol:cholesterol (p<0.05 for ATORVA or EZE/ATORVA vs. PBO and vs. EZE; p<0.05 for EZE/ATORVA vs. ATORVA for lathosterol only; Table in FIG. 13). EZE increased both ratios (p<0.05 vs. PBO for lathosterol; increases were not significant for desmosterol).

EZE/ATORVA significantly reduces concentrations of LDL-C, total cholesterol, and triglycerides and increases concentrations of HDL-C relative to ATORVA.

EZE, ATORVA and EZE/ATORVA all produce significant changes in LDL-C, total cholesterol, and triglycerides relative to baseline.

EZE/ATORVA significantly reduces concentrations of phytosterols, cholesterol precursors, and LDL-C.

In patients with primary hypercholesterolemia EZE significantly reduced plasma concentrations of the phytosterols (sitosterol and campesterol) and increased plasma concentrations of the cholesterol precursors/synthesis markers (lathosterol and desmosterol).

ATORVA significantly increased plasma phytosterol concentrations and the ratios of sitosterol:cholesterol and campesterol:cholesterol most likely due to decreases in cholesterol concentrations, reflecting inhibition of cholesterol synthesis.

EZE/ATORVA significantly reduced plasma concentrations of both phytosterols and sterol markers of cholesterol synthesis relative to PBO.

These findings are consistent with the known complementary mechanisms of action of EZE and ATORVA on sterol metabolism. EZE blocks the intestinal uptake of cholesterol and non-cholesterol sterols which in turn leads to reduced plasma concentrations and a compensatory increase in endogenous cholesterol synthesis. ATORVA inhibits cholesterol synthesis in the liver but increases the concentrations of phytosterols. Thus, through dual inhibition of sterol absorption and cholesterol synthesis, EZE/ATORVA coadministration produces large, multitargeted reductions in cholesterol and non-cholesterol sterols in patients with primary hypercholesterolemia.

EZE significantly lowered concentrations of phytosterols and increased concentrations of cholesterol precursors. ATORVA (pooled) significantly lowered concentrations of cholesterol precursors and increased phytosterol:cholesterol ratios. The increase in absolute concentration of sitosterol ranged from 7.2% to 24.9% and was significantly greater than placebo for the 40 and 80 mg doses (p<0.05). EZE+ATORVA significantly lowered concentrations of phytosterols and cholesterol precursors, and the phytosterol:cholesterol ratios.

TABLE 8

| | | | | | | | EZE 10 mg + | |
|---|---|---|---|---|---|---|---|---|
| | | | | | ATORVA | | ATORVA | |
| | Pbo | | EZE 10 mg | | (pooled) | | (pooled) | |
| | (n = 41) | | (n = 39) | | (n = 160) | | (n = 157) | |
| | ^{a}% chng | | ^{a}% chng | | ^{a}% chng | | ^{a}% chng | |
| Sterol | LSmean | ^{b}Ratio | LSmean | ^{b}Ratio | LSmean | ^{b}Ratio | LSmean | ^{b}Ratio |
| Sitosterol | 6.7 | 0.8 | −53.8* | −70.1* | 16.1^{†} | 77.4*^{†} | −49.4*^{‡} | −24.8*^{†‡} |
| Campesterol | 7.3 | 5.2 | −58.2* | −151* | 10.1^{†} | 128*^{†} | −59.3*^{‡} | −88.9*^{†‡} |
| Lathosterol | 6.0 | −0.0 | 34.5* | 56.2* | −69.0*^{†} | −69.8*^{†} | −62.4*^{†} | −51.2*^{†‡} |
| Desmosterol | 6.6 | 9.1 | 20.6* | 38.1 | −55.4*^{†} | −34.5*^{†} | −55.1*^{†} | −35.1*^{†} |

^{a}Absolute sterol concentrations(mg/dL).
^{b}Noncholesterols:Cholesterol Ratio: Mean change(10² mmol/mol)
*p < 0.05 vs placebo;
^{†}p < 0.05 vs EZE 10;
^{‡}p < 0.05 vs ATORVA(pooled)

These results indicate that treatment with ATORVA increases phytosterols while decreasing cholesterol precursors concentrations in a manner that appears to be dose related. EZE+ATORVA lowered both plasma cholesterol and phytosterol concentrations, presumably through dual inhibition of hepatic cholesterol synthesis and intestinal cholesterol/phytosterol absorption.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, Genbank Accession Numbers and protocols are cited throughout this application, the disclosures of which are incorporated herein by reference in their entireties for all purposes.

We claim:

1. A method for characterizing a subject's risk profile of developing a future cardiovascular event, comprising:
(a) obtaining a level of at least one material selected from the group consisting of a phytosterol, a cholesterol precursor and a stanol in a subject having no history of clinically evident coronary heart disease prior to obtaining the level;
(b) comparing the level of the material to a predetermined material value; and
(c) characterizing the subject's risk profile of developing a future cardiovascular event based upon the level of the material in comparison to the predetermined material value.

2. A method for characterizing a subject's risk profile of developing a future myocardial infarction, comprising:
(a) obtaining a level of at least one material selected from the group consisting of a phytosterol, a cholesterol precursor and a stanol in a subject;
(b) comparing the level of the material to a predetermined material value; and
(c) characterizing the subject's risk profile of developing a future myocardial infarction based upon the level of the material in comparison to the predetermined material value.

3. A method for characterizing a subject's risk profile of developing a future cardiovascular disorder associated with atherosclerotic disease, comprising:
(a) obtaining a level of at least one material selected from the group consisting of a phytosterol, a cholesterol precursor and a stanol in a subject;
(b) comparing the level of the material to a predetermined material value; and
(c) characterizing the subject's risk profile of developing a future cardiovascular disorder associated with atherosclerotic disease based upon the level of the material in comparison to the predetermined material value.

4. A method for characterizing a subject's risk profile of developing a future cardiovascular disorder associated with atherosclerotic disease, comprising:
(a) obtaining a level of at least one material selected from the group consisting of a phytosterol, a cholesterol precursor and a stanol in a subject;
(b) comparing the level of the material to a predetermined material value to establish a first risk value;
(c) obtaining a level of cholesterol in the subject;
(d) comparing the level of the cholesterol to a second predetermined cholesterol value to establish a second risk value; and
(e) characterizing the subject's risk profile of developing a future cardiovascular disorder associated with atherosclerotic disease based upon a combination of the first risk value and the second risk value.

5. A method for evaluating the likelihood that a subject will benefit from treatment with an sterol absorption inhibitor for reducing risk of a vascular disorder, comprising:
(a) obtaining a level of at least one material selected from the group consisting of a phytosterol, a cholesterol precursor and a stanol in a subject; and
(b) comparing the level of the material to a predetermined material value, wherein the level of the material in comparison to the predetermined material value is indicative of whether the subject will benefit from treatment with the sterol absorption inhibitor.

6. The method according to claim 1, wherein the subject is an apparently healthy, non-smoker.

7. The method according to claim 1, wherein the subject has no previous history of myocardial infarction.

8. The method according to claim 1, wherein the phytosterol is selected from the group consisting of sitosterol, campesterol, stigmasterol and avenosterol.

9. The method according to claim 8, wherein the phytosterol is sitosterol.

10. The method according to claim 8, wherein the phytosterol is campesterol.

11. The method according to claim 1, wherein the predetermined material value is greater than about 4.5 micromoles per liter of plasma, blood, serum or tissue.

12. The method according to claim 1, wherein the predetermined material value is greater than about 5.0 micromoles per liter of plasma, blood, serum or tissue.

13. The method according to claim 1, wherein the predetermined material value is greater than about 5.25 micromoles per liter of plasma, blood, serum or tissue.

14. The method according to claim 1, wherein the predetermined material value is greater than about 7.0 micromoles per liter of plasma, blood, serum or tissue.

15. The method according to claim 11, wherein the phytosterol is sitosterol.

16. The method according to claim 1, wherein the predetermined material value is a plurality of predetermined value ranges and the comparing step (b) comprises determining into which of the predetermined value ranges the subject's material level falls.

17. The method according to claim 16, wherein one of the plurality of value ranges is less than about 5.25 micromoles per liter of plasma or tissue and another of the plurality of value ranges is greater than about 5.25 micromoles per liter of plasma or tissue, and wherein the comparing step (b) comprises determining in which of the plurality of value ranges the subject's level falls.

18. The method according to claim 1, wherein the cardiovascular event or disorder or the vascular disorder is stroke.

19. The method according to claim 1, wherein the cardiovascular event or disorder or the vascular disorder is myocardial infarction.

20. The method according to claim 5, wherein the sterol absorption inhibitor is selected from the group consisting of:
(a) a sterol absorption inhibitor represented by Formula (I): 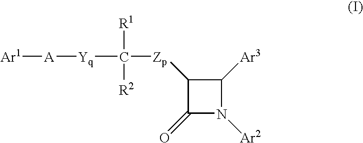
or isomers thereof, or pharmaceutically acceptable salts or solvates of the compounds of Formula (I) or of the isomers thereof, wherein in Formula (I):
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(di-lower alkyl)-;
A is —O—, —S—, —S(O)— or —S(O)₂—;
R¹ is selected from the group consisting of —OR⁶, —O(CO)R⁶, —O(CO)OR⁹ and —O(CO)NR⁶R⁷;
R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO²-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl;
(b) a sterol absorption inhibitor represented by Formula (II): 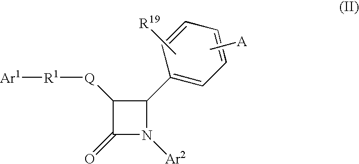
or isomers thereof, or pharmaceutically acceptable salts or solvates of the compounds of Formula (II) or of the isomers thereof, wherein in Formula (II):
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 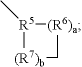
R¹ is selected from the group consisting of —(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or
—S(O)₀₋₂-e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is 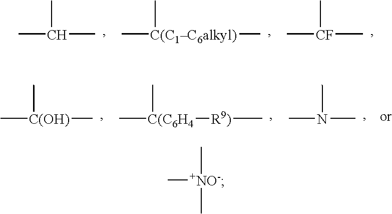
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or
R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R¹ also can be: 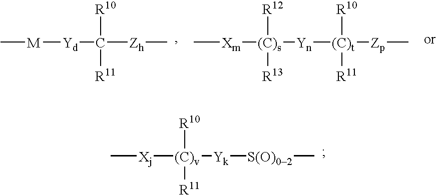
M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or
R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 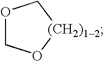
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 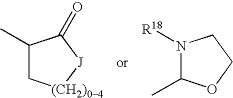
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR1⁴, —O(CO)NR¹⁴R¹⁵, —NR¹⁴R¹⁵, —NR¹⁴(CO)R¹⁵, —NR¹⁴(CO)OR¹⁶, —NR¹⁴(Co)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy;
(c) a sterol absorption inhibitor represented by Formula (III): 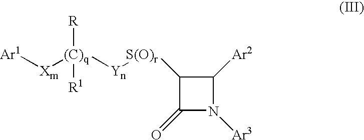
or isomers thereof, or pharmaceutically acceptable salts or solvates of the compounds of Formula (III) or of the isomers thereof, wherein in Formula (III):
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(di-lower alkyl)-;
R is —OR⁶, —O(CO)R⁶, —O(CO)OR⁹ or —O(CO)NR⁶R⁷;
R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)R⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂ and halogen;
(d) a sterol absorption inhibitor represented by Formula (IV): 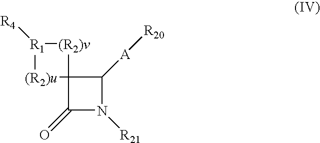
or isomers thereof, or pharmaceutically acceptable salts or solvates of the compounds of Formula (IV) or of the isomers thereof, wherein in Formula (IV):
R₁ is 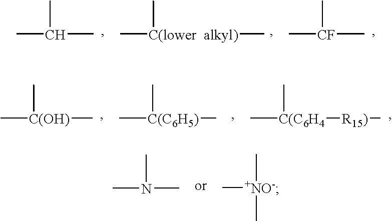
R₂ and R₃ are independently selected from the group consisting of:
—CH₂—, —CH(lower alkyl)-, —C(di-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;
u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;
R₄ is selected from B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ—V—(C₂-C₆ alkenylene)- or
B—(C₂-C₆ alkenylene)-V-(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or
R₁ and R₄ together form the group B—CH═C—;
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 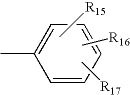
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R¹², R₁₀C(O)(lower alkylenyloxy)-, N(R₈)(R₉)C(O)(lower alkylenyloxy)- and 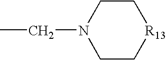
for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;
R₇ is 1-3 groups independently selected from the group consisting of lower alkyl, lower alkoxy, —COOH, NO₂, —N(R₈)(R₉), OH, and halogeno;
R₈ and R₉ are independently selected from H or lower alkyl;
R₁₀ is selected from lower alkyl, phenyl, R₇-phenyl, benzyl or R₇-benzyl;
R₁₁ is selected from OH, lower alkyl, phenyl, benzyl, R₇-phenyl or R₇-benzyl;
R₁₂ is selected from H, OH, alkoxy, phenoxy, benzyloxy, 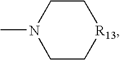
N(R₈)(R₉), lower alkyl, phenyl or R₇-phenyl;
R₁₃ is selected from —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above;
(e) a sterol absorption inhibitor represented by Formula (VA) or Formula (VB): 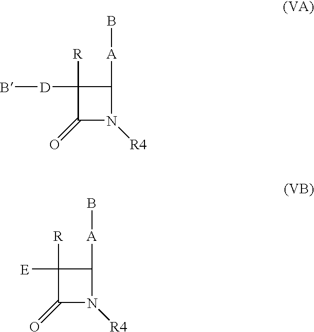
or isomers thereof, or pharmaceutically acceptable salts or solvates of the compounds of Formula (VA) or (VB) or of the isomers thereof, wherein in Formulae (VA) or (VB):
A is —CH═CH—, —C≡C— or —(CH₂)ₚ— wherein p is 0, 1 or 2;
B is 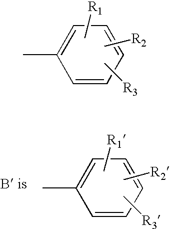
D is —(CH₂)ₘC(O)— or —(CH₂)_{q}— wherein m is 1, 2, 3 or 4 and q is 2, 3 or 4;
E is C₁₀ to C₂₀ alkyl or —C(O)—(C₉ to C₁₉)-alkyl, wherein the alkyl is straight or branched, saturated or containing one or more double bonds;
R is hydrogen, C₁-C₁₅ alkyl, straight or branched, saturated or containing one or more double bonds, or B—(CH₂)ᵣ—, wherein r is 0, 1, 2, or 3;
R₁, R₂, R₃, R_{1′}, R_{2′}, and R_{3′}are independently selected from the group consisting of hydrogen, lower alkyl, lower alkoxy, carboxy, NO₂, NH₂, OH, halogeno, lower alkylamino, di-lower alkylamino, —NHC(O)OR₅, R₆O₂SNH— and —S(O)₂NH₂;
R₄ is 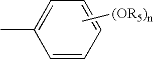
wherein n is 0, 1, 2 or 3;
R₅ is lower alkyl; and
R₆ is OH, lower alkyl, phenyl, benzyl or substituted phenyl, wherein the substituents are 1-3 groups independently selected from the group consisting of lower alkyl, lower alkoxy, carboxy, NO₂, NH₂, OH, halogeno, lower alkylamino and di-lower alkylamino;
(f) a sterol absorption inhibitor represented by Formula (VI): 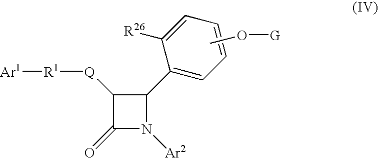
or isomers thereof, or pharmaceutically acceptable salts or solvates of the compounds of Formula (VI) or of the isomers thereof, wherein in Formula (VI):
R₂₆ is H or OG¹;
G and G¹ are independently selected from the group consisting of H, 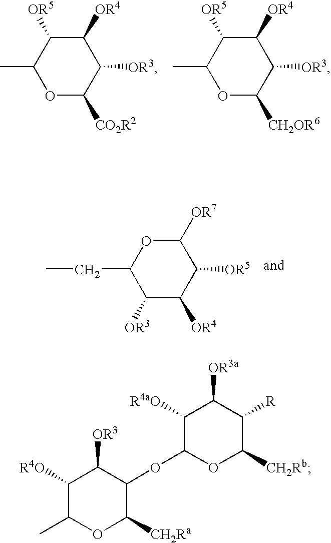
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy and —W—R³⁰;
wherein W is independently selected from the group consisting of —NH—C(O), —O—C(O)—, —O—C(O)—N(R³³)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl; R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or
R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 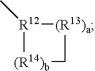
and
R¹ is selected from the group consisting of:
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 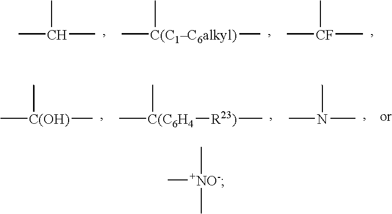
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹¹, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴'s can be the same or different;
and when Q is a bond, R¹ also can be: 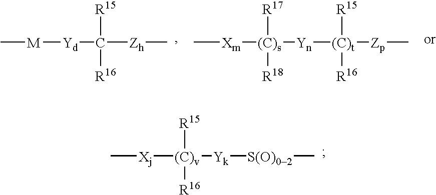
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²⁰, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
his 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 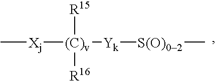
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy;
(g) a sterol absorption inhibitor represented by Formula (VII): 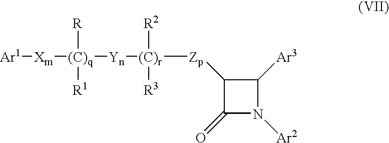
or isomers thereof, or pharmaceutically acceptable salts or solvates of the compounds of Formula (VII) or of the isomers thereof, wherein in Formula (VII):
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(di-lower alkyl)-;
R² and R⁶ are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently 0, 1, 2, 3 or 4;
provided that at least one of q and r is 1, and the sum of m, n, p, q and r is 1, 2, 3, 4, 5 or 6; and
provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl; and
(h) a sterol absorption inhibitor represented by Formula (IX): 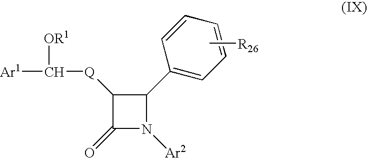
or isomers thereof, or pharmaceutically acceptable salts or solvates of the compounds of Formula (IX) or of the isomers thereof, wherein in Formula (IX):
R²⁶ is selected from the group consisting of:
a) OH;
b) OCH₃;
c) fluorine and
d) chlorine.
R¹ is selected from the group consisting of H, 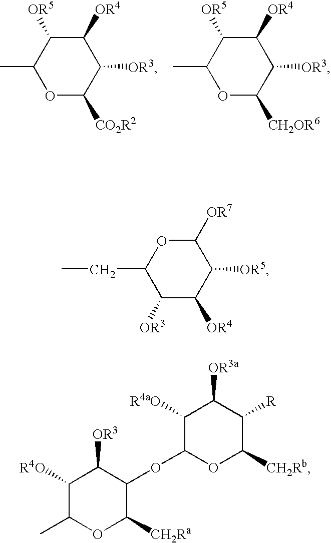
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone, forms the spiro group 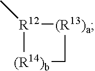
R¹² is 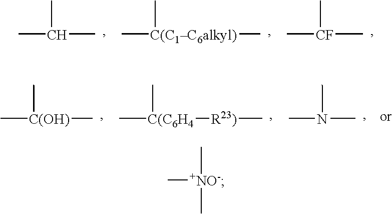
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²′, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R², —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰—OH and halogeno; and
R²⁵ is H, —OH or (C₁-C₆)alkoxy.

21. The method of claim 20, wherein the sterol absorption inhibitor is represented by Formula (VIII): 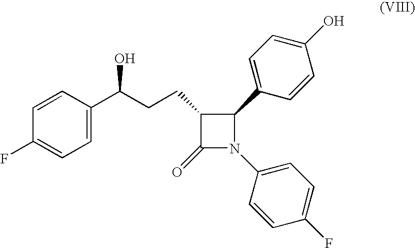
or pharmaceutically acceptable salts or solvates thereof.

22. The method according to claim 5, wherein the sterol absorption inhibitor is ezetimibe.
